(19) United States
(12) Patent Application Publication
(10) Pub. No.: US 2024/0327927 A1
(43) Pub. Date: Oct. 3, 2024

(54) ACTIVE SURVEILLANCE AND RISK STRATIFICATION FOR PROSTATE CANCER

(71) Applicant: IMMUNIS.AI, Inc., Corvallis, OR (US)

(72) Inventors: Amin I. Kassis, Corvallis, OR (US); Geoffrey Erickson, Corvallis, OR (US); Ricard Henao, Corvallis, OR (US); Kirk Wojno, Corvallis, OR (US); Harry Stylli, Corvallis, OR (US); Leander Van Neste, Corvallis, OR (US)

(21) Appl. No.: 18/618,857

(22) Filed: Mar. 27, 2024

Related U.S. Application Data
(60) Provisional application No. 63/539,739, filed on Sep. 21, 2023.
(60) Provisional application No. 63/492,289, filed on Mar. 27, 2023.

(51) Int. Cl. C12Q 1/6886

(52) U.S. Cl. CPC C12Q 1/6886; C12Q 2600/118; C12Q 2600/158

(57) ABSTRACT

The present disclosure relates to compositions, methods, and systems useful for assessing whether a patient with prostate cancer is a candidate for active surveillance of the prostate cancer, as well as compositions, methods, and systems for assessing and identifying prostate cancer. The disclosure provides algorithm-based assays comprising subtraction-normalized immunocyte signature profiling from a sample obtained from a prostate cancer patient. Measurement of expression of signature markers identify on an individual basis, via an active surveillance risk score, prostate cancer patients that are to enter, continue, or stop an active surveillance pathway.

### CROSS-REFERENCE TO RELATED APPLICATIONS

This application claims priority to U.S. Provisional Patent Application No. 63/539,739, filed Sep. 21, 2023, and to U.S. Provisional Patent Application No. 63/492,289, filed Mar. 27, 2023, the contents of each of which are incorporated by reference in their entirety.

### FIELD OF THE APPLICATION

The present disclosure relates to compositions, methods, and systems useful for assessing whether a patient with prostate cancer is a candidate for active surveillance of the prostate cancer, as well as compositions, methods, and systems for assessing and identifying prostate cancer. The disclosure provides algorithm-based assays comprising subtraction-normalized immunocyte signature profiling from a sample obtained from a prostate cancer patient. Measurement of expression of signature markers identify on an individual basis, via an active surveillance risk score, prostate cancer patients that are to enter, continue, or stop an active surveillance pathway. Compositions, methods, and systems of the disclosure find use in clinical and research settings, for example, within the fields of biology, immunology, medicine, and oncology.

### BACKGROUND

In men, prostate cancer (PCa) remains the most frequently diagnosed cancer and second leading cause of cancer death, despite the availability of diagnostic and screening tools (1). Prostate-specific antigen (PSA) screening changes over the time impacted by the US Preventive Services Task Force (USPSTF) recommendation in 2012 has changed the characteristics of study cohorts needed to be considered when evaluating results of large trials (3). It has become increasingly clear that low-grade, low-volume PCa poses very limited risk for the patient, both in terms of morbidity and mortality(5). The National Comprehensive Cancer Network (NCCN) categorizes a subset of men diagnosed with PCa as having (very) low risk (6). Even for men that do not have the lowest risk profile, the urology community considers favorable intermediate risk patients as potential active surveillance (AS) candidates (7,8). Multiparametric MRI (mpMRI) has been shown to be a useful but imperfect tool at distinguishing men with higher risk disease. Nonetheless, the only way to diagnose and monitor men with PCa has been through prostate biopsy, typically sampling twelve cores, or less than 1%, of the entire prostate (9). Because of the limited sampling, disease can either be missed, or aggressive foci can go undetected. Studies following patients over time clearly demonstrate the diagnostic limitation due to biopsy sampling error (10). In most settings, monitoring now consists of mpMRI and periodic biopsies. In a study performed at Johns Hopkins, patients with indolent cancer were monitored with annual follow-up biopsies. Despite the rigorous selection criteria, 18.5% of the patients were upgraded over time, resulting in the detection of aggressive disease warranting treatment (11). However, in other institutions, upgrading rates as high as 40% have been observed (12), indicating that additional tools are required to identify men with occult high-risk disease early on.

Due to frequent detection of early-stage and low-grade disease, many men with indolent PCa have been subjected to unnecessary invasive treatments. In the last decade, AS protocols became a mainstream means to follow men with low-risk cancer (13). These programs are generally recommended for men with at least ten years of life expectancy, as there is no evidence-based support for treating early-stage PCa in men with shorter life expectancy (14). Unfortunately, there is no perfect standard to identify men that truly belong on an AS protocol. The NCCN has identified several risk groups ranging from very low to very high, with guidance on how patients should be managed relative to their corresponding risk group (6). Patients with very low, low, and favorable intermediate disease could be managed without immediate treatment (7,8,15). While classical biomarkers have been used in this decision tree, i.e. PSA and PSA density, other parameters, e.g. Gleason grade group (GG), number of positive cores, and tissue-based genomic profiling signatures are purely driven by the biopsy result and suffer from the same limitations of under-sampling and differences in pathology interpretation (16). In addition to the number of cancer-positive cores, the main risk factor remains the biopsy GG.

### SUMMARY OF THE DISCLOSURE

The present disclosure is based on the discovery of a genetic signature that is useful for identifying whether a patient with prostate cancer should enter, continue, and/or stop active surveillance.

The disclosure provides molecular assays that involve measurement of expression level(s) of a plurality of genes or gene subsets from a biological sample obtained from a prostate cancer patient, and analysis of the measured expression levels to provide information concerning whether the patient with prostate cancer should enter, continue, and/or stop active surveillance. The disclosure provides molecular assays that involve measurement of expression level(s) of a plurality of genes or gene subsets from a biological sample obtained to identify an active surveillance risk score (ASRS) for a prostate cancer patient. For example, patients may be stratified using expression level(s) of a plurality of genes, positively or negatively, with positive clinical outcome of prostate cancer, or with a prognostic factor. In an exemplary embodiment, the prognostic factor is Gleason score.

The present disclosure provides, in one embodiment, a method of predicting and/or determining whether a patient with prostate cancer should enter, continue, and/or stop active surveillance comprising determination of a level of a plurality of RNA transcripts, or an expression product thereof, in a biological sample obtained from the patient, wherein the RNA transcript, or its expression product, is selected from the genes shown in FIG. 6. In some embodiments, the method comprises calculating a quantitative score (e.g., an active surveillance risk score (ASRS)) for the prostate cancer patient by weighting the level of the one or more RNA transcripts or an expression product thereof, by their contribution to a clinical outcome and predicting and/or determining whether the patient with prostate cancer should enter, continue, and/or stop active surveillance based on the quantitative score. In one embodiment, the active surveillance risk score (ASRS) of low or average risk may identify a patient with prostate cancer as a candidate for active surveillance. In one embodiment, the one or more RNA transcripts, or an expression product thereof, is selected from ENTREP1, KIR2DL4, KIF2C, BICC1, ROR2, LOC124904706, DUSP2, LOC122455342, ST6GALNAC2, POU2F3, LOC124908063, DKK3, DKK2, KLRF1, MYO18B, KLRC2, MATN2, FCER1A, GPRC5C, CLCN4, H3-5, LOC105374736, EPB41L4A, KCNJ10, SYNM, MEIS3, FOXD1, IQSEC3, NEBL, PLXNA3, LILRB5, PF4, SIGLEC17P, TPBG, RORB, CSMD1, SCGB3A2, OR1F1, CA2, ITGB3, FST, PPBP, SLC35F3, PPP1R14C, RNF217, ROBO1, LINC01644, LRRC77P, TMEM171, BCAS1, PDE5A, DPYSL4, NAV3, LINC01819, PRUNE2, IGLV3-12, SH3BGRL2, TUBB1, COLEC12, CDK14, KRT1, TMEM255A, SLC44A5, LARGE1, ITGA11, C1QC, WNT5B, DNAH10, COL19A1, XKR9, CELSR1, MEG3, MYOM2, ADAMTS2, TCL1A, ADORA3, ZNF890P, SPIB, DYNLT5, SH3RF2, TRIM58, PTPRB, FZD6, ADGRB3, KREMEN1, SYCE1, OR2W3, NYAP2, UTS2, DOC2B, SORCS2, FSIP2, GRIP1, HLA-DRB6, RAMP3, FCRL1, LOC101929563, LINC01918, CPE, GLIS3, S100B, HBEGF, SFTPB, PTPRG, CFAP95, ORM1, ANXA9, PSAD or a combination thereof.

In some embodiments the present disclosure provides a diagnostic method for determining whether a patient with prostate cancer should enter, continue, and/or stop active surveillance, the method comprising measuring the level of expression of a plurality of biomarker genes (e.g., 2 or more, 5 or more, 10 or more, 20 or more, 30 or more, 40 or more, 50 or more, 60 or more, 70 or more, 80 or more, 90 or more biomarker genes) comprising ENTREP1, KIR2DL4, KIF2C, BICC1, ROR2, LOC124904706, DUSP2, LOC122455342, ST6GALNAC2, POU2F3, LOC124908063, DKK3, DKK2, KLRF1, MYO18B, KLRC2, MATN2, FCER1A, GPRC5C, CLCN4, H3-5, LOC105374736, EPB41L4A, KCNJ10, SYNM, MEIS3, FOXD1, IQSEC3, NEBL, PLXNA3, LILRB5, PF4, SIGLEC17P, TPBG, RORB, CSMD1, SCGB3A2, OR1F1, CA2, ITGB3, FST, PPBP, SLC35F3, PPP1R14C, RNF217, ROBO1, LINC01644, LRRC77P, TMEM171, BCAS1, PDE5A, DPYSL4, NAV3, LINC01819, PRUNE2, IGLV3-12, SH3BGRL2, TUBB1, COLEC12, CDK14, KRT1, TMEM255A, SLC44A5, LARGE1, ITGA11, C1QC, WNT5B, DNAH10, COL19A1, XKR9, CELSR1, MEG3, MYOM2, ADAMTS2, TCL1A, ADORA3, ZNF890P, SPIB, DYNLT5, SH3RF2, TRIM58, PTPRB, FZD6, ADGRB3, KREMEN1, SYCE1, OR2W3, NYAP2, UTS2, DOC2B, SORCS2, FSIP2, GRIP1, HLA-DRB6, RAMP3, FCRL1, LOC101929563, LINC01918, CPE, GLIS3, S100B, HBEGF, SFTPB, PTPRG, CFAP95, ORM1, and ANXA9, or a combination thereof, in a biological sample obtained from a subject; and calculating an active surveillance risk score (ASRS) based on the level of expression of the plurality of biomarkers. In some embodiments, the patient's prostate specific antigen (PSA) density (PSAD) is also measured. In some embodiments, the active surveillance risk score (ASRS) indicates the probability that the subject with prostate cancer harbors (e.g., currently harbors or will harbor in the future) aggressive prostate cancer. In one embodiment, an active surveillance risk score (ASRS) of very low risk or an ASRS of low/average risk identifies a patient with prostate cancer as a candidate for active surveillance (e.g., that enters or remains on active surveillance (e.g., instead of receiving treatment for prostate cancer)). In some embodiments, an ASRS score of very low risk or an ASRS of low/average risk indicates that the patient with prostate cancer, in consultation with his physician, may want to consider entering active surveillance. In some embodiments, an ASRS score of high risk identifies the patient with prostate cancer is not a candidate for active surveillance (e.g., that the patient instead should receive prostate cancer treatment). In some embodiments, an ASRS score of very low risk or an ASRS of low/average risk indicates that it is desirable for the patient with prostate cancer, in consultation with his physician, to start, remain on, and/or continue active surveillance. In some embodiments, an ASRS score of high risk indicates that it is desirable that the patient with prostate cancer, in consultation with his physician, stops active surveillance. In some embodiments, a prostate cancer patient is classified with an ASRS of very low risk if the patient has a 25 (+/−25) % probability of harboring aggressive prostate cancer (e.g., determined by the methods disclosed herein). For example, in some embodiments, a prostate cancer patient with a 0-5%, 5-10%, 10-15%, 15-20%, 20-25%, 25-30%, 30-35% or more probability of harboring aggressive prostate cancer determined by the methods disclosed herein (e.g., from a biological sample from the prostate cancer patient determining the normalized expression of 2 or more, 5 or more, 10 or more, 20 or more, 30 or more, 40 or more, 50 or more, 60 or more, 70 or more, 80 or more, 90 or more biomarker genes of FIG. 6 from phagocytic and non-phagocytic cells of the sample using the log ratio of phagocytic (e.g., macrophage) cell expression level divided by non-phagocytic (e.g., non-macrophage) cell expression level) is classified with and/or assigned an ASRS of very low risk. In a preferred embodiment, a prostate cancer patient is classified with an ASRS of very low risk if the patient has a 15 (+/−15) % probability of harboring aggressive prostate cancer (e.g., determined by the methods disclosed herein). In some embodiments, a prostate cancer patient is classified with an ASRS of low/average risk if the patient has a 50 (+/−25) % probability of harboring aggressive prostate cancer (e.g., determined by the methods disclosed herein). For example, in some embodiments, a prostate cancer patient with a 35-40%, 40-45%, 45-50%, 50-55%, 55-60%, 60-65%, 65-70% or more probability of harboring aggressive prostate cancer determined by the methods disclosed herein (e.g., from a biological sample from the prostate cancer patient determining the normalized expression of 2 or more, 5 or more, 10 or more, 20 or more, 30 or more, 40 or more, 50 or more, 60 or more, 70 or more, 80 or more, 90 or more biomarker genes of FIG. 6 from phagocytic and non-phagocytic cells of the sample using the log ratio of macrophage cell expression level divided by non-macrophage cell expression level) is classified with and/or assigned an ASRS of low/average risk. In a preferred embodiment, a prostate cancer patient is classified with an ASRS of low/average risk if the patient has a 50 (+/−20) % probability of harboring aggressive prostate cancer (e.g., determined by the methods disclosed herein). In some embodiments, a prostate cancer patient is classified with an ASRS of high risk if the patient has a 75 (+/−25) % probability of harboring aggressive prostate cancer (e.g., determined by the methods disclosed herein). For example, in some embodiments, a prostate cancer patient with a 70-75%, 75-80%, 80-85%, 85-90%, 90-95%, 96%, 97%, 98% or more probability of harboring aggressive prostate cancer determined by the methods disclosed herein (e.g., from a biological sample from the prostate cancer patient determining the normalized expression of 2 or more, 5 or more, 10 or more, 20 or more, 30 or more, 40 or more, 50 or more, 60 or more, 70 or more, 80 or more, 90 or more biomarker genes of FIG. 6 from phagocytic and non-phagocytic cells of the sample using the log ratio of macrophage cell expression level divided by non-macrophage cell expression level) is classified with and/or assigned an ASRS of high risk. In a preferred embodiment, a prostate cancer patient is classified with an ASRS of high risk if the patient has a 85 (+/−15) % probability of harboring aggressive prostate cancer (e.g., determined by the methods disclosed herein).

In some embodiments, the decision to treat or not to treat a prostate cancer patient utilizes an ASRS determined by a method of the disclosure. The decision to treat or not to treat prostate cancer may be a collective decision (e.g., a shared decision making process) between a physician and the prostate cancer patient. In some embodiments, the shared decision making considers only the ASRS determined by a method disclosed herein. For example, in some embodiments, when an ASRS is determined for a prostate cancer patient to fall within very low risk, or in the mid- to low-range of low/intermediate risk (e.g., classified with an ASRS of 45, 44, 43, 42, 41, 40, 39, 38, 37, 36, 35, 34, 33, 32, 31 or lower percent probability of harboring aggressive prostate cancer determined by a method disclosed herein), the decision is made to place the patient on active surveillance or to continue active surveillance and to forego definitive treatment of the prostate cancer. In another embodiment, when an ASRS is determined for a prostate cancer patient to fall within high risk or in the mid- to high-range of low/intermediate risk (e.g., classified with an ASRS of 65, 66, 67, 68, 69 or higher percent probability of harboring aggressive prostate cancer determined by a method disclosed herein), the decision is made that the patient would benefit from treating the prostate cancer or that the patient is not a candidate for active surveillance or that the patient should not remain on active surveillance. In some embodiments, an ASRS determined by a method of the present disclosure is utilized in combination with one or more different quantitative metrics (e.g., age) and/or one or more qualitative metrics (e.g., a prostate cancer patient's subjective risk tolerance or life priorities) in a shared decision making process between a physician and the prostate cancer patient to determine an individualized plan for the prostate cancer patient. In some embodiments, an ASRS is utilized in combination with one or more other guidelines/recommendations for prostate cancer care and/or management including, but not limited to, those provided by the American Cancer Society (ACS), the National Comprehensive Cancer Network (NCCN), the American Urological Association (AUA)/Society of Urologic Oncology (SUO), the U.S. Preventive Services Task Force (USPSTF), the European Society for Medical Oncology (ESMO), and the European Association of Urology/European Association of Nuclear Medicine/European Society for Radiotherapy and Oncology/European Society of Urogenital Radiology/International Society of Urological Pathology/International Society of Geriatric Oncology (EAU/EANM/ESTRO/ESUR/ISUP/SIOG).

In some embodiments, a method of the disclosure (e.g., a diagnostic method, a method of analyzing a biological sample, a method of measuring a panel of biomarkers, and/or a method of measuring the level of a marker) is performed while the patient with prostate cancer is under active surveillance for prostate cancer, is not under surveillance for prostate cancer, is undergoing treatment for prostate cancer, and/or is post treatment for prostate cancer. In some embodiments, the method is performed before and/or after the subject undergoes radical prostatectomy. In some embodiments, the method is performed before and/or after radiation therapy. In some embodiments, the method is performed before and/or after surgery. In some embodiments, the method is performed during, before and/or after chemotherapy or other treatment for prostate cancer. In some embodiments, the patient with prostate cancer has a family history of prostate or other type of cancer (e.g., breast, colon, lung, esophageal, or other type of cancer). In some embodiments, the patient with prostate cancer is known to be susceptible to cancer (e.g., possesses one or more mutations (e.g., BRCA1 and/or BRCA2 mutations)).

In some embodiments, the prostate cancer in the subject is adenocarcinoma, small cell prostate cancer, non-small cell prostate cancer, neuroendocrine prostate cancer, or metastatic castration resistant prostate cancer.

In another embodiment, the method further comprises predicting survival of a subject before and/or after undergoing treatment of prostate cancer.

In some embodiments, the biological sample obtained from the subject is a blood sample. In some embodiments, the biological sample is a liquid biopsy (e.g., blood, urine, or other body fluid). In some embodiments, the blood sample and/or liquid biopsy comprises phagocytic and non-phagocytic cells. In some embodiments, the phagocytic cells are CD14+. The disclosure is not limited by the type of CD14+ cells. Indeed, any type of CD14+ cells may be used including, but not limited to, macrophages, neutrophils, and/or dendritic cells. In some embodiments, the non-phagocytic cells are T cells, B cells, null cells, basophils, or mixtures thereof. In some embodiments, the non-phagocytic cells are CD2+ T cells. In some embodiments, nucleic acids comprising biomarker gene sequences are isolated from the phagocytic and non-phagocytic cells of the biological sample, and/or purified, and/or amplified prior to analysis.

In some embodiments, the expression level of biomarker nucleic acids is determined by a sequencing technique selected from direct sequencing, RNA sequencing, whole transcriptome shotgun sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD® sequencing, MS-PET sequencing, mass spectrometry, and/or a combination thereof.

In some embodiments, gene/biomarker expression level (e.g., copy number) in phagocytic (e.g., CD14+) cells and/or non-phagocytic (e.g., CD2+) cells is determined using massively parallel sequencing (that is, simultaneously or in rapid succession sequencing any of at least 100, 1000, 10,000, 100,000, 1 million, 10 million, 100 million, 1 billion or more polynucleotide molecules). Various sequencing methods useful for measuring gene/biomarker nucleic acid expression levels (e.g., copy number) in phagocytic (e.g., CD14+) cells and/or non-phagocytic (e.g., CD2+) cells include, but are not limited to, Next generation sequencing, RNA-Seq (Illumina), massively-parallel sequencing, high-throughput sequencing, sequencing using PacBio, SOLiD, Single Molecule Sequencing by Synthesis (SMSS) (Helicos), Ion Torrent, pyrosequencing, sequencing-by-synthesis, single-molecule sequencing, nanopore sequencing, semiconductor sequencing, sequencing-by-ligation, sequencing-by-hybridization, Digital Gene Expression (Helicos), Clonal Single Molecule Array (Solexa), shotgun sequencing, Maxam-Gilbert or Sanger sequencing, primer walking, or Nanopore platforms and any other sequencing methods known in the art.

In some embodiments, the expression level of biomarker nucleic acids is determined by polymerase chain reaction (PCR) analysis, sequencing analysis, electrophoretic analysis, restriction fragment length polymorphism (RFLP) analysis, Northern blot analysis, quantitative PCR, reverse-transcriptase-PCR analysis (RT-PCR), allele specific oligonucleotide hybridization analysis, comparative genomic hybridization, heteroduplex mobility assay (HMA), single strand conformational polymorphism (SSCP), denaturing gradient gel electrophisis (DGGE), RNAase mismatch analysis, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), surface plasmon resonance, Southern blot analysis, in situ hybridization, fluorescence in situ hybridization (FISH), chromogenic in situ hybridization (CISH), immunohistochemistry (IHC), microarray, comparative genomic hybridization, karyotyping, multiplex ligation-dependent probe amplification (MLPA), Quantitative Multiplex PCR of Short Fluorescent Fragments (QMPSF), microscopy, methylation specific PCR (MSP) assay, Hpaii tiny fragment Enrichment by Ligation-mediated PCR (HELP) assay, radioactive acetate labeling assays, colorimetric DNA acetylation assay, chromatin immunoprecipitation combined with microarray (ChiP-on-chip) assay, restriction landmark genomic scanning, Methylated DNA immunoprecipitation (MeDIP), molecular break light assay for DNA adenine methyltransferase activity, chromatographic separation, methylation-sensitive restriction enzyme analysis, bisulfite-driven conversion of non-methylated cytosine to uracil, methyl-binding PCR analysis, and/or a combination thereof.

In another aspect, the disclosure includes a method for treating a subject for prostate cancer, the method comprising determining whether or not the patient with prostate cancer should enter, continue, and/or stop active surveillance, as described herein; and administering treatment to the subject if the subject is not identified as a candidate for active surveillance. The patient with prostate cancer may be administered a cancer treatment comprising, for example, surgery, radiation therapy, chemotherapy, immunotherapy, hormone therapy (e.g., ADT) or biologic therapy, or any combination thereof.

In another aspect, the disclosure includes a kit for measuring expression levels of biomarker genes for identifying whether a patient with prostate cancer should enter, continue, and/or stop active surveillance, as described herein. The kit may include one or more agents for measuring expression levels of biomarker genes (e.g., hybridization probes, PCR primers, or microarray), a container for holding a biological sample (e.g., blood sample) isolated from a subject (e.g., a human or non-human patient with prostate cancer) for testing, and printed instructions for reacting the agents with the biological sample or a portion of the biological sample to determine whether or not the patient with prostate cancer should enter, continue, and/or stop active surveillance. The agents may be packaged in separate containers. The kit may further comprise one or more control reference samples or other reagents for measuring gene expression (e.g., reagents for performing PCR, RT-PCR, microarray analysis, a Northern blot, or SAGE).

In one embodiment, the kit comprises agents for measuring levels of expression of biomarker genes comprising ENTREP1, KIR2DL4, KIF2C, BICC1, ROR2, LOC124904706, DUSP2, LOC122455342, ST6GALNAC2, POU2F3, LOC124908063, DKK3, DKK2, KLRF1, MYO18B, KLRC2, MATN2, FCER1A, GPRC5C, CLCN4, H3-5, LOC105374736, EPB41L4A, KCNJ10, SYNM, MEIS3, FOXD1, IQSEC3, NEBL, PLXNA3, LILRB5, PF4, SIGLEC17P, TPBG, RORB, CSMD1, SCGB3A2, OR1F1, CA2, ITGB3, FST, PPBP, SLC35F3, PPP1R14C, RNF217, ROBO1, LINC01644, LRRC77P, TMEM171, BCAS1, PDE5A, DPYSL4, NAV3, LINC01819, PRUNE2, IGLV3-12, SH3BGRL2, TUBB1, COLEC12, CDK14, KRT1, TMEM255A, SLC44A5, LARGE1, ITGA11, C1QC, WNT5B, DNAH10, COL19A1, XKR9, CELSR1, MEG3, MYOM2, ADAMTS2, TCL1A, ADORA3, ZNF890P, SPIB, DYNLT5, SH3RF2, TRIM58, PTPRB, FZD6, ADGRB3, KREMEN1, SYCE1, OR2W3, NYAP2, UTS2, DOC2B, SORCS2, FSIP2, GRIP1, HLA-DRB6, RAMP3, FCRL1, LOC101929563, LINC01918, CPE, GLIS3, S100B, HBEGF, SFTPB, PTPRG, CFAP95, ORM1, ANXA9, PSA or a combination thereof.

The significance of the expression levels of one or more biomarker genes may be evaluated using, for example, a T-test, P-value, S (Olmogorov Smirnov) P-value, accuracy, accuracy P-value, positive predictive value (PPV), negative predictive value (NPV), sensitivity, specificity, precision, AUC, AUC P-value (Auc.pvalue), Wilcoxon Test P-value, Median Fold Difference (MFD), Kaplan Meier (KM) curves, survival AUC (survAUC), Kaplan Meier P-value (KM P-value), Univariable Analysis Odds Ratio P-value (uvaORPval), multivariable analysis Odds Ratio P-value (mvaORPval), Univariable Analysis Hazard Ratio P-value (uvaHRPval) and Multivariable Analysis Hazard Ratio P-value (mvaHRPval). The significance of the expression level of the one or more targets may be based on two or more metrics selected from the group comprising AUC, AUC P-value (Auc.pvalue), Wilcoxon Test P-value, Median Fold Difference (MFD), Kaplan Meier (KM) curves, survival AUC (survAUC), Univariable Analysis Odds Ratio P-value (uvaORPval), multivariable analysis Odds Ratio P-value (mvaORPval), Kaplan Meier P-value (KM P-value), Univariable Analysis Hazard Ratio P-value (uvaHRPval), and/or Multivariable Analysis Hazard Ratio P-value (mvaHRPval).

In another aspect, the disclosure provides a computer implemented method for identifying whether a patient with prostate cancer should enter, continue, and/or stop active surveillance, the computer performing steps comprising receiving inputted subject data comprising values for the levels of expression of a plurality of biomarker genes selected from ENTREP1, KIR2DL4, KIF2C, BICC1, ROR2, LOC124904706, DUSP2, LOC122455342, ST6GALNAC2, POU2F3, LOC124908063, DKK3, DKK2, KLRF1, MYO18B, KLRC2, MATN2, FCER1A, GPRC5C, CLCN4, H3-5, LOC105374736, EPB41L4A, KCNJ10, SYNM, MEIS3, FOXD1, IQSEC3, NEBL, PLXNA3, LILRB5, PF4, SIGLEC17P, TPBG, RORB, CSMD1, SCGB3A2, OR1F1, CA2, ITGB3, FST, PPBP, SLC35F3, PPP1R14C, RNF217, ROBO1, LINC01644, LRRC77P, TMEM171, BCAS1, PDE5A, DPYSL4, NAV3, LINC01819, PRUNE2, IGLV3-12, SH3BGRL2, TUBB1, COLEC12, CDK14, KRT1, TMEM255A, SLC44A5, LARGE1, ITGA11, C1QC, WNT5B, DNAH10, COL19A1, XKR9, CELSR1, MEG3, MYOM2, ADAMTS2, TCL1A, ADORA3, ZNF890P, SPIB, DYNLT5, SH3RF2, TRIM58, PTPRB, FZD6, ADGRB3, KREMEN1, SYCE1, OR2W3, NYAP2, UTS2, DOC2B, SORCS2, FSIP2, GRIP1, HLA-DRB6, RAMP3, FCRL1, LOC101929563, LINC01918, CPE, GLIS3, S100B, HBEGF, SFTPB, PTPRG, CFAP95, ORM1, ANXA9, and/or a combination thereof in a biological sample from the subject; normalizing the expression level of each of the selected markers via determining the log ratio of phagocytic cell expression (e.g., CD14+ cell expression) over non-phagocytic cell expression (e.g., CD2+ cell expression); and calculating an active surveillance risk score (ASRS) based on the patient's normalized expression levels, and displaying and/or reporting information regarding the ASRS. In some embodiments, the patient's PSAD is measured and utilized as inputted subject data. In some embodiments, the reported information is useful for determining whether a patient with prostate cancer should enter active surveillance (e.g., if the displayed and/or reported ASRS of the patient is very low risk or low/average risk). In some embodiments, the reported information is useful for determining whether a patient with prostate cancer should not enter active surveillance (e.g., if the displayed and/or reported ASRS of the patient is high risk). In some embodiments, the reported information is useful for determining whether a patient with prostate cancer should continue active surveillance (e.g., if the displayed and/or reported ASRS of the patient is very low risk or low/average risk). In some embodiments, the reported information is useful for determining whether a patient with prostate cancer should stop active surveillance (e.g., if the displayed and/or reported ASRS of the patient is high risk).

In another aspect, the disclosure provides a diagnostic system for determining whether a patient with prostate cancer should enter, continue, and/or stop active surveillance, the diagnostic system comprising a storage component (memory) for storing data, wherein the storage component has instructions for calculating an active surveillance risk score (ASRS) for the subject stored therein; a computer processor for processing data, wherein the computer processor is coupled to the storage component and configured to execute the instructions stored in the storage component in order to receive subject data and analyze subject data according to one or more algorithms; and a display component for displaying information (e.g., information regarding the ASRS and/or clinical recommendation and/or diagnosis of the subject).

In one embodiment, the present disclosure provides a method comprising measuring the level of expression of a plurality of biomarker genes comprising ENTREP1, KIR2DL4, KIF2C, BICC1, ROR2, LOC124904706, DUSP2, LOC122455342, ST6GALNAC2, POU2F3, LOC124908063, DKK3, DKK2, KLRF1, MYO18B, KLRC2, MATN2, FCER1A, GPRC5C, CLCN4, H3-5, LOC105374736, EPB41L4A, KCNJ10, SYNM, MEIS3, FOXD1, IQSEC3, NEBL, PLXNA3, LILRB5, PF4, SIGLEC17P, TPBG, RORB, CSMD1, SCGB3A2, OR1F1, CA2, ITGB3, FST, PPBP, SLC35F3, PPP1R14C, RNF217, ROBO1, LINC01644, LRRC77P, TMEM171, BCAS1, PDE5A, DPYSL4, NAV3, LINC01819, PRUNE2, IGLV3-12, SH3BGRL2, TUBB1, COLEC12, CDK14, KRT1, TMEM255A, SLC44A5, LARGE1, ITGA11, C1QC, WNT5B, DNAH10, COL19A1, XKR9, CELSR1, MEG3, MYOM2, ADAMTS2, TCL1A, ADORA3, ZNF890P, SPIB, DYNLT5, SH3RF2, TRIM58, PTPRB, FZD6, ADGRB3, KREMEN1, SYCE1, OR2W3, NYAP2, UTS2, DOC2B, SORCS2, FSIP2, GRIP1, HLA-DRB6, RAMP3, FCRL1, LOC101929563, LINC01918, CPE, GLIS3, S100B, HBEGF, SFTPB, PTPRG, CFAP95, ORM1, ANXA9, PSAD or a combination thereof in a biological sample from a subject; calculating an active surveillance risk score (ASRS) based on the level of expression of the plurality of biomarkers; and determining whether or not to administer a treatment (e.g., surgery, chemotherapy, radiation therapy, immunotherapy, and/or other prostate cancer treatment) to the subject (e.g., based upon the ASRS). In some embodiments, the subject is undergoing prostate cancer treatment. In a further aspect, the method is performed after treatment of the subject with a prostate cancer treatment. In some embodiments, the prostate cancer is adenocarcinoma, small cell prostate cancer, neuroendocrine prostate cancer or metastatic castration resistant prostate cancer. In some embodiments, the method is performed after the subject undergoes radical prostatectomy. In some embodiments, the treatment is a cancer treatment comprising surgery, radiation therapy, chemotherapy, immunotherapy, biologic therapy, or any combination thereof.

In another aspect, the disclosure provides methods of measuring a panel of biomarkers in a subject with prostate cancer, the method comprising: obtaining a biological sample from the subject with prostate cancer; determining a measurement for the panel of biomarkers in the biological sample, wherein the panel of biomarkers comprise two or more biomarkers selected from FIG. 6, and wherein the measurement comprises measuring a level of each of the biomarkers in the panel. In some embodiments, the panel of biomarkers comprises three, four, five, six, seven, eight, nine, ten, eleven, twelve, thirteen, fourteen, 14-20, 20-40, 40-60, 60-80, 80-100 or more biomarkers selected from FIG. 6. In some embodiments, the panel of biomarkers comprise ten or more biomarkers selected from FIG. 6. In some embodiments, the panel of biomarkers comprise twenty or more biomarkers selected from FIG. 6. In some embodiments, the panel of biomarkers comprises fifty or more biomarkers selected from FIG. 6. In some embodiments, the method further comprises obtaining one or more clinical data from the subject with prostate cancer selected from age, race, digital rectal exam (DRE), prostate volume, prostate density, family history, total prostate-specific antigen (PSA), PSA density (PSAD), tumor stage, tumor grade, tumor size, tumor visual characteristics, tumor growth, tumor thickness, tumor progression, tumor metastasis, tumor distribution within the body, odor, molecular pathology, genomics, and/or tumor angiograms. In some embodiments, the one or more clinical data from the subject comprises the prostate-specific antigen (PSA) density of the patient with prostate cancer. In some embodiments, the one or more clinical data from the subject comprises the age of the patient with prostate cancer. In some embodiments, the biological sample comprises CD2⁺ cells and/or CD14⁺ cells.

In another aspect, the disclosure provides methods for measuring the level of a marker in a sample from a subject, the method comprising the steps of: a) measuring the levels of ten or more markers selected from the group ENTREP1, KIR2DL4, KIF2C, BICC1, ROR2, LOC124904706, DUSP2, LOC122455342, ST6GALNAC2, POU2F3, LOC124908063, DKK3, DKK2, KLRF1, MYO18B, KLRC2, MATN2, FCER1A, GPRC5C, CLCN4, H3-5, LOC105374736, EPB41L4A, KCNJ10, SYNM, MEIS3, FOXD1, IQSEC3, NEBL, PLXNA3, LILRB5, PF4, SIGLEC17P, TPBG, RORB, CSMD1, SCGB3A2, OR1F1, CA2, ITGB3, FST, PPBP, SLC35F3, PPP1R14C, RNF217, ROBO1, LINC01644, LRRC77P, TMEM171, BCAS1, PDE5A, DPYSL4, NAV3, LINC01819, PRUNE2, IGLV3-12, SH3BGRL2, TUBB1, COLEC12, CDK14, KRT1, TMEM255A, SLC44A5, LARGE1, ITGA11, C1QC, WNT5B, DNAH10, COL19A1, XKR9, CELSR1, MEG3, MYOM2, ADAMTS2, TCL1A, ADORA3, ZNF890P, SPIB, DYNLT5, SH3RF2, TRIM58, PTPRB, FZD6, ADGRB3, KREMEN1, SYCE1, OR2W3, NYAP2, UTS2, DOC2B, SORCS2, FSIP2, GRIP1, HLA-DRB6, RAMP3, FCRL1, LOC101929563, LINC01918, CPE, GLIS3, S100B, HBEGF, SFTPB, PTPRG, CFAP95, ORM1, and ANXA9 in a population of the subject's phagocytic cells; b) measuring the levels of the ten or more selected markers in a population of the subject's non-phagocytic cells; wherein the measuring the levels of the ten or more selected markers occurs prior to the subject receiving treatment for prostate cancer. In some embodiments, the measuring the levels of the ten or more selected markers comprises measuring gene expression levels. In some embodiments, the gene expression levels are measured by a sequencing technique selected from the group consisting of direct sequencing, RNA sequencing, whole transcriptome shotgun sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD® sequencing, MS-PET sequencing, mass spectrometry, and a combination thereof. In some embodiments, the gene expression levels are measured by polymerase chain reaction (PCR) analysis, sequencing analysis, electrophoretic analysis, restriction fragment length polymorphism (RFLP) analysis, Northern blot analysis, quantitative PCR, reverse-transcriptase-PCR analysis (RT-PCR), allele specific oligonucleotide hybridization analysis, comparative genomic hybridization, heteroduplex mobility assay (HMA), single strand conformational polymorphism (SSCP), denaturing gradient gel electrophisis (DGGE), RNAase mismatch analysis, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), surface plasmon resonance, Southern blot analysis, in situ hybridization, fluorescence in situ hybridization (FISH), chromogenic in situ hybridization (CISH), immunohistochemistry (IHC), microarray, comparative genomic hybridization, karyotyping, multiplex ligation-dependent probe amplification (MLPA), Quantitative Multiplex PCR of Short Fluorescent Fragments (QMPSF), microscopy, methylation specific PCR (MSP) assay, Hpaii tiny fragment Enrichment by Ligation-mediated PCR (HELP) assay, radioactive acetate labeling assays, colorimetric DNA acetylation assay, chromatin immunoprecipitation combined with microarray (ChiP-on-chip) assay, restriction landmark genomic scanning, Methylated DNA immunoprecipitation (MeDIP), molecular break light assay for DNA adenine methyltransferase activity, chromatographic separation, methylation-sensitive restriction enzyme analysis, bisulfite-driven conversion of non-methylated cytosine to uracil, methyl-binding PCR analysis, or a combination thereof. In some embodiments, the gene expression levels are measured by an amplification assay or a sequencing assay. In some embodiments, the non-phagocytic cells are T cells, B cells, null cells, basophils, or mixtures thereof. In some embodiments, the method further comprises measuring at least one standard parameter associated with prostate cancer. In some embodiments, the standard parameter is selected from the group consisting of tumor stage, tumor grade, tumor size, tumor visual characteristics, tumor growth, tumor thickness, tumor progression, tumor metastasis, tumor distribution within the body, odor, molecular pathology, genomics, or tumor angiograms. In some embodiments, measuring the levels of the ten or more selected markers comprises measuring the levels at a first time point and measuring the levels at a second time point, wherein at least the first time point occurs prior to the subject receiving treatment for prostate cancer. In some embodiments, the second time point occurs after the subject has been placed on active surveillance of the prostate cancer for a period of time (e.g., 3, 6, 12, 18, 24, 30, 36, 48 or more months). In some embodiments, the second time point occurs after the subject receives treatment for prostate cancer. In some embodiments, the phagocytic cells are macrophages. In some embodiments, the methods further comprise obtaining one or more clinical data from the subject with prostate cancer selected from the group consisting of age, race, digital rectal exam (DRE), prostate volume, prostate density, family history, total prostate-specific antigen (PSA), PSA density (PSAD), tumor stage, tumor grade, tumor size, tumor visual characteristics, tumor growth, tumor thickness, tumor progression, tumor metastasis, tumor distribution within the body, odor, molecular pathology, genomics, and/or tumor angiograms. In some embodiments, the one or more clinical data from the subject comprises the prostate-specific antigen (PSA) density of the patient with prostate cancer. In some embodiments, the one or more clinical data from the subject comprises the age of the patient with prostate cancer.

In some embodiments, the disclosure provides a method for identifying, assessing and/or predicting the aggressiveness or indolence of cancer (e.g., prostate cancer) in a subject (e.g., a subject suspected of having cancer, a subject diagnosed with a cancer, or a subject at risk for cancer). In some embodiments, the disclosure provides a method for identifying, assessing and/or predicting the aggressiveness or indolence of prostate cancer.

In some embodiments, the disclosure provides a method of measuring a panel of biomarkers in a subject comprising obtaining a biological sample from the subject; determining a measurement for the panel of biomarkers in the biological sample, wherein the panel of biomarkers comprise a plurality of biomarker genes (e.g., 2 or more, 5 or more, 10 or more, 20 or more, 30 or more, 40 or more, 50 or more, 60 or more, 70 or more, 80 or more, 90 or more biomarker genes) comprising ENTREP1, KIR2DL4, KIF2C, BICC1, ROR2, LOC124904706, DUSP2, LOC122455342, ST6GALNAC2, POU2F3, LOC124908063, DKK3, DKK2, KLRF1, MYO18B, KLRC2, MATN2, FCER1A, GPRC5C, CLCN4, H3-5, LOC105374736, EPB41L4A, KCNJ10, SYNM, MEIS3, FOXD1, IQSEC3, NEBL, PLXNA3, LILRB5, PF4, SIGLEC17P, TPBG, RORB, CSMD1, SCGB3A2, OR1F1, CA2, ITGB3, FST, PPBP, SLC35F3, PPP1R14C, RNF217, ROBO1, LINC01644, LRRC77P, TMEM171, BCAS1, PDE5A, DPYSL4, NAV3, LINC01819, PRUNE2, IGLV3-12, SH3BGRL2, TUBB1, COLEC12, CDK14, KRT1, TMEM255A, SLC44A5, LARGE1, ITGA11, C1QC, WNT5B, DNAH10, COL19A1, XKR9, CELSR1, MEG3, MYOM2, ADAMTS2, TCL1A, ADORA3, ZNF890P, SPIB, DYNLT5, SH3RF2, TRIM58, PTPRB, FZD6, ADGRB3, KREMEN1, SYCE1, OR2W3, NYAP2, UTS2, DOC2B, SORCS2, FSIP2, GRIP1, HLA-DRB6, RAMP3, FCRL1, LOC101929563, LINC01918, CPE, GLIS3, S100B, HBEGF, SFTPB, PTPRG, CFAP95, ORM1, and ANXA9, or a combination thereof, and wherein the measurement comprises measuring a level of each of the biomarkers in the panel. In some embodiments, measuring the panel of biomarkers in the subject identifies, assesses, and/or predicts the aggressiveness or indolence of cancer (e.g., prostate cancer) in a subject (e.g., a subject suspected of having cancer, a subject diagnosed with a cancer, or a subject at risk for cancer). In some embodiments, the biological sample comprises CD2⁺ cells and/or CD14⁺ cells. In one embodiment, determining a measurement for the panel of biomarkers in the biological sample comprises measuring a level of each of the biomarkers in the panel in CD2⁺ cells and/or CD14⁺ cells. In one embodiment, the method further comprises obtaining one or more clinical data from the subject selected from the group consisting of age, race, digital rectal exam (DRE), prostate density, and total prostate-specific antigen (PSA). The disclosure is not limited by the type of clinical data obtained and/or used. Additional examples of clinical data include, but are not limited to, tumor stage, tumor grade, tumor size, tumor visual characteristics, tumor growth, tumor thickness, tumor progression, tumor metastasis, tumor distribution within the body, odor, molecular pathology, genomics, and/or tumor angiograms. In some embodiments, the one or more clinical data are used as clinical covariates and concatenated with the biomarker levels and input into a sparse rank regression model/algorithm (e.g., in order to identify, assess, and/or predict the aggressiveness or indolence of cancer (e.g., prostate cancer) in a subject). In one embodiment, the algorithm provides a cancer (e.g., prostate cancer) aggressiveness index value (e.g., 0, 1, 2, 3, or 4) that identifies and characterizes cancer in a subject (e.g., scaled such that a value of 0 characterizes the absence of cancer in the subject ranging to a value of 4 that characterizes the presence of highly aggressive cancer in the subject). In some embodiments, the aggressiveness index value (e.g., 0, 1, 2, 3, or 4) is used alone or in combination with an active surveillance risk score (ASRS) disclosed herein (e.g., in order to determine whether or not the subject should enter or remain on active surveillance or receive further assessment (e.g., biopsy) or receive treatment (e.g., chemotherapy, surgery, radiation therapy, or other treatment known in the art) for prostate cancer. In some embodiments, measuring a level of each of the biomarkers in the panel comprises measuring gene expression levels. The disclosure is not limited by how gene expression levels are measured. Indeed, any means of measuring gene expression levels may be used including, but not limited to, polymerase chain reaction (PCR) analysis, sequencing analysis, electrophoretic analysis, restriction fragment length polymorphism (RFLP) analysis, Northern blot analysis, quantitative PCR, reverse-transcriptase-PCR analysis (RT-PCR), allele-specific oligonucleotide hybridization analysis, comparative genomic hybridization, heteroduplex mobility assay (HMA), single strand conformational polymorphism (SSCP), denaturing gradient gel electrophoresis (DGGE), RNAase mismatch analysis, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), surface plasmon resonance, Southern blot analysis, in situ hybridization, fluorescence in situ hybridization (FISH), chromogenic in situ hybridization (CISH), immunohistochemistry (IHC), microarray, comparative genomic hybridization, karyotyping, multiplex ligation-dependent probe amplification (MLPA), Quantitative Multiplex PCR of Short Fluorescent Fragments (QMPSF), microscopy, methylation specific PCR (MSP) assay, HpaII tiny fragment Enrichment by Ligation-mediated PCR (HELP) assay, radioactive acetate labeling assays, colorimetric DNA acetylation assay, chromatin immunoprecipitation combined with microarray (ChIP-on-chip) assay, restriction landmark genomic scanning, Methylated DNA immunoprecipitation (MeDIP), molecular break light assay for DNA adenine methyltransferase activity, chromatographic separation, methylation-sensitive restriction enzyme analysis, bisulfite-driven conversion of non-methylated cytosine to uracil, methyl-binding PCR analysis, or a combination thereof. In some embodiments, gene expression levels are measured by a sequencing technique such as, but not limited to, direct sequencing, RNA sequencing, whole transcriptome shotgun sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, mass spectrometry, and a combination thereof. In some embodiments, measuring a level of each of the biomarkers in the panel comprises measuring protein expression levels. The disclosure is not limited to any particular method of measuring protein expression levels. Exemplary methods of measuring protein expression levels include, but are not limited to, an immunohistochemistry assay, an enzyme-linked immunosorbent assay (ELISA), in situ hybridization, chromatography, liquid chromatography, size exclusion chromatography, high performance liquid chromatography (HPLC), gas chromatography, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), radioimmunoassays, microscopy, microfluidic chip-based assays, surface plasmon resonance, sequencing, Western blotting assay, or a combination thereof. In some embodiments, measuring a level of each of the biomarkers in the panel comprises measuring by a qualitative assay, a quantitative assay, or a combination thereof. Exemplary quantitative assays include, but are not limited to, sequencing, direct sequencing, RNA sequencing, whole transcriptome shotgun sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), polymerase chain reaction (PCR) analysis, quantitative PCR, real-time PCR, fluorescence assay, colorimetric assay, chemiluminescent assay, or a combination thereof. In some embodiments, the subject is a human.

In another aspect, the disclosure provides methods for detecting or diagnosing prostate cancer by using at least one or more (e.g., two, three, four, five, six, seven, eight, nine, ten, eleven, twelve, thirteen, fourteen, fifteen or more) markers selected from ENTREP1, KIR2DL4, KIF2C, BICC1, ROR2, LOC124904706, DUSP2, LOC122455342, ST6GALNAC2, POU2F3, LOC124908063, DKK3, DKK2, KLRF1, MYO18B, KLRC2, MATN2, FCER1A, GPRC5C, CLCN4, H3-5, LOC105374736, EPB41L4A, KCNJ10, SYNM, MEIS3, FOXD1, IQSEC3, NEBL, PLXNA3, LILRB5, PF4, SIGLEC17P, TPBG, RORB, CSMD1, SCGB3A2, OR1F1, CA2, ITGB3, FST, PPBP, SLC35F3, PPP1R14C, RNF217, ROBO1, LINC01644, LRRC77P, TMEM171, BCAS1, PDE5A, DPYSL4, NAV3, LINC01819, PRUNE2, IGLV3-12, SH3BGRL2, TUBB1, COLEC12, CDK14, KRT1, TMEM255A, SLC44A5, LARGE1, ITGA11, C1QC, WNT5B, DNAH10, COL19A1, XKR9, CELSR1, MEG3, MYOM2, ADAMTS2, TCL1A, ADORA3, ZNF890P, SPIB, DYNLT5, SH3RF2, TRIM58, PTPRB, FZD6, ADGRB3, KREMEN1, SYCE1, OR2W3, NYAP2, UTS2, DOC2B, SORCS2, FSIP2, GRIP1, HLA-DRB6, RAMP3, FCRL1, LOC101929563, LINC01918, CPE, GLIS3, S100B, HBEGF, SFTPB, PTPRG, CFAP95, ORM1, and ANXA9, or a combination thereof. Levels (e.g., gene expression levels, protein expression levels, or activity levels) of the selected markers may be measured from phagocytic cells (e.g., macrophages, monocytes, dendritic cells, and/or neutrophils) and from non-phagocytic cells (e.g., T cells), from a subject. Such levels then can be compared, e.g., the levels of the selected markers in the phagocytic cells and in the non-phagocytic cells to identify one or more differences between the measured levels, indicating whether the subject has prostate cancer. The identified difference(s) can also be used for assessing the risk of developing prostate cancer, prognosing prostate cancer, monitoring prostate cancer progression or regression, assessing the efficacy of a treatment for prostate cancer, or identifying a compound capable of ameliorating or treating prostate cancer.

In yet another aspect, the levels of the selected markers in the phagocytic cells may be compared to the levels of the selected markers in a control (e.g., a normal or healthy control subject, or a normal or healthy cell from the subject) to identify one or more differences between the measured levels, indicating whether the subject has prostate cancer, the prognosis of the cancer and the monitoring of the cancer. The identified difference(s) can also be used for assessing the risk of developing prostate cancer, prognosing prostate cancer, monitoring prostate cancer progression or regression, assessing the efficacy of a treatment for prostate cancer, or identifying a compound capable of ameliorating or treating prostate cancer.

In some embodiments, the disclosure provides a method for diagnosing or aiding in the diagnosis of prostate cancer in a subject, the method comprising the steps of: a) measuring the levels of one or more (e.g., two, three, four, five, six, seven, eight, nine, ten, eleven, twelve, thirteen, fourteen, fifteen or more) markers selected from ENTREP1, KIR2DL4, KIF2C, BICC1, ROR2, LOC124904706, DUSP2, LOC122455342, ST6GALNAC2, POU2F3, LOC124908063, DKK3, DKK2, KLRF1, MYO18B, KLRC2, MATN2, FCER1A, GPRC5C, CLCN4, H3-5, LOC105374736, EPB41L4A, KCNJ10, SYNM, MEIS3, FOXD1, IQSEC3, NEBL, PLXNA3, LILRB5, PF4, SIGLEC17P, TPBG, RORB, CSMD1, SCGB3A2, OR1F1, CA2, ITGB3, FST, PPBP, SLC35F3, PPP1R14C, RNF217, ROBO1, LINC01644, LRRC77P, TMEM171, BCAS1, PDE5A, DPYSL4, NAV3, LINC01819, PRUNE2, IGLV3-12, SH3BGRL2, TUBB1, COLEC12, CDK14, KRT1, TMEM255A, SLC44A5, LARGE1, ITGA11, C1QC, WNT5B, DNAH10, COL19A1, XKR9, CELSR1, MEG3, MYOM2, ADAMTS2, TCL1A, ADORA3, ZNF890P, SPIB, DYNLT5, SH3RF2, TRIM58, PTPRB, FZD6, ADGRB3, KREMEN1, SYCE1, OR2W3, NYAP2, UTS2, DOC2B, SORCS2, FSIP2, GRIP1, HLA-DRB6, RAMP3, FCRL1, LOC101929563, LINC01918, CPE, GLIS3, S100B, HBEGF, SFTPB, PTPRG, CFAP95, ORM1, and ANXA9, or a combination thereof, in a population of the subject's macrophage or monocyte cells; b) measuring the levels of the one or more selected markers in a population of the subject's non-phagocytic cells (e.g., T cells); and c) identifying a difference between the measured levels of the one or more selected markers in steps a) and b), wherein the identified difference indicates that the subject has prostate cancer.

In some embodiments, the disclosure provides a method for diagnosing or aiding in the diagnosis of prostate cancer in a subject, the method comprising the steps of: a) measuring the levels of one or more markers selected from ENTREP1, KIR2DL4, KIF2C, BICC1, ROR2, LOC124904706, DUSP2, LOC122455342, ST6GALNAC2, POU2F3, LOC124908063, DKK3, DKK2, KLRF1, MYO18B, KLRC2, MATN2, FCER1A, GPRC5C, CLCN4, H3-5, LOC105374736, EPB41L4A, KCNJ10, SYNM, MEIS3, FOXD1, IQSEC3, NEBL, PLXNA3, LILRB5, PF4, SIGLEC17P, TPBG, RORB, CSMD1, SCGB3A2, OR1F1, CA2, ITGB3, FST, PPBP, SLC35F3, PPP1R14C, RNF217, ROBO1, LINC01644, LRRC77P, TMEM171, BCAS1, PDE5A, DPYSL4, NAV3, LINC01819, PRUNE2, IGLV3-12, SH3BGRL2, TUBB1, COLEC12, CDK14, KRT1, TMEM255A, SLC44A5, LARGE1, ITGA11, C1QC, WNT5B, DNAH10, COL19A1, XKR9, CELSR1, MEG3, MYOM2, ADAMTS2, TCL1A, ADORA3, ZNF890P, SPIB, DYNLT5, SH3RF2, TRIM58, PTPRB, FZD6, ADGRB3, KREMEN1, SYCE1, OR2W3, NYAP2, UTS2, DOC2B, SORCS2, FSIP2, GRIP1, HLA-DRB6, RAMP3, FCRL1, LOC101929563, LINC01918, CPE, GLIS3, S100B, HBEGF, SFTPB, PTPRG, CFAP95, ORM1, and ANXA9, or a combination thereof. in a population of the subject's macrophage or monocyte cells; b) measuring the levels of the one or more markers selected from ENTREP1, KIR2DL4, KIF2C, BICC1, ROR2, LOC124904706, DUSP2, LOC122455342, ST6GALNAC2, POU2F3, LOC124908063, DKK3, DKK2, KLRF1, MYO18B, KLRC2, MATN2, FCER1A, GPRC5C, CLCN4, H3-5, LOC105374736, EPB41L4A, KCNJ10, SYNM, MEIS3, FOXD1, IQSEC3, NEBL, PLXNA3, LILRB5, PF4, SIGLEC17P, TPBG, RORB, CSMD1, SCGB3A2, OR1F1, CA2, ITGB3, FST, PPBP, SLC35F3, PPP1R14C, RNF217, ROBO1, LINC01644, LRRC77P, TMEM171, BCAS1, PDE5A, DPYSL4, NAV3, LINC01819, PRUNE2, IGLV3-12, SH3BGRL2, TUBB1, COLEC12, CDK14, KRT1, TMEM255A, SLC44A5, LARGE1, ITGA11, C1QC, WNT5B, DNAH10, COL19A1, XKR9, CELSR1, MEG3, MYOM2, ADAMTS2, TCL1A, ADORA3, ZNF890P, SPIB, DYNLT5, SH3RF2, TRIM58, PTPRB, FZD6, ADGRB3, KREMEN1, SYCE1, OR2W3, NYAP2, UTS2, DOC2B, SORCS2, FSIP2, GRIP1, HLA-DRB6, RAMP3, FCRL1, LOC101929563, LINC01918, CPE, GLIS3, S100B, HBEGF, SFTPB, PTPRG, CFAP95, ORM1, and ANXA9, or a combination thereof in a population of the subject's non-phagocytic cells (e.g., T cells) or from a cell free component from the subject; and c) identifying a difference between the measured levels of the one or more selected markers in steps a) and b), wherein the identified difference indicates that the subject has prostate cancer.

In some embodiments, the disclosure provides a method of identifying a patient with prostate cancer as a candidate for active surveillance of the prostate cancer comprising: a) obtaining a blood sample from the patient with prostate cancer; b) isolating CD14+ cells and CD2+ cells from the blood sample; c) determining the gene expression level of 10 or more biomarkers selected from the group consisting of ENTREP1, KIR2DL4, KIF2C, BICC1, ROR2, LOC124904706, DUSP2, LOC122455342, ST6GALNAC2, POU2F3, LOC124908063, DKK3, DKK2, KLRF1, MYO18B, KLRC2, MATN2, FCER1A, GPRC5C, CLCN4, H3-5, LOC105374736, EPB41L4A, KCNJ10, SYNM, MEIS3, FOXD1, IQSEC3, NEBL, PLXNA3, LILRB5, PF4, SIGLEC17P, TPBG, RORB, CSMD1, SCGB3A2, OR1F1, CA2, ITGB3, FST, PPBP, SLC35F3, PPP1R14C, RNF217, ROBO1, LINC01644, LRRC77P, TMEM171, BCAS1, PDE5A, DPYSL4, NAV3, LINC01819, PRUNE2, IGLV3-12, SH3BGRL2, TUBB1, COLEC12, CDK14, KRT1, TMEM255A, SLC44A5, LARGE1, ITGA11, C1QC, WNT5B, DNAH10, COL19A1, XKR9, CELSR1, MEG3, MYOM2, ADAMTS2, TCL1A, ADORA3, ZNF890P, SPIB, DYNLT5, SH3RF2, TRIM58, PTPRB, FZD6, ADGRB3, KREMEN1, SYCE1, OR2W3, NYAP2, UTS2, DOC2B, SORCS2, FSIP2, GRIP1, HLA-DRB6, RAMP3, FCRL1, LOC101929563, LINC01918, CPE, GLIS3, S100B, HBEGF, SFTPB, PTPRG, CFAP95, ORM1, and ANXA9 in the CD14+ cells; d) determining the expression level of the 10 or more selected biomarkers in the CD2+ cells; e) normalizing the expression level of each of the selected biomarkers by determining the log ratio of CD14+ over CD2+ expression levels; f) using the normalized expression level of each of the 10 or more selected biomarkers of step (e) to calculate an active surveillance risk score (ASRS) representing the probability that the patient is harboring aggressive prostate cancer; and g) categorizing the patient into a group selected from very low risk, low/average risk, and high risk based on the patient's ASRS. In some embodiments, when the ASRS is very low risk or low/average risk the patient with prostate cancer is identified as a candidate for active surveillance of the prostate cancer. In some embodiments, the identified candidate for active surveillance of the prostate cancer enters active surveillance of the prostate cancer or remains on active surveillance of the prostate cancer. In some embodiments, the method further comprises obtaining one or more clinical data from the patient with prostate cancer selected from the group consisting of age, race, digital rectal exam (DRE), prostate volume, prostate density, family history, total prostate-specific antigen (PSA), PSA density (PSAD), tumor stage, tumor grade, tumor size, tumor visual characteristics, tumor growth, tumor thickness, tumor progression, tumor metastasis, tumor distribution within the body, odor, molecular pathology, genomics, and/or tumor angiograms. In some embodiments, calculating an active surveillance risk score (ASRS) of step (f) further comprises utilizing the prostate-specific antigen (PSA) density of the patient with prostate cancer and/or the age of the patient with prostate cancer. The method of determining gene expression levels is not limited to any particular method. Indeed, any method disclosed herein or known in the art may be used to determine gene expression level. In some embodiments, the method further comprising (h) creating a report containing the active surveillance risk score (ASRS). In some embodiments, the report containing the active surveillance risk score (ASRS) is utilized by the patient with prostate cancer and the patient's physician in a shared decision making process to determine the course of treatment or surveillance of the patient's prostate cancer. In a further embodiment, the report containing the active surveillance risk score (ASRS) is utilized by the patient with prostate cancer and the patient's physician in combination with one or more other guidelines or recommendations in a shared decision making process to determine the course of treatment or surveillance of the patient's prostate cancer.

The disclosure also provides a method of characterizing prostate cancer aggressiveness in a patient with prostate cancer comprising: a) obtaining a blood sample from the patient with prostate cancer; b) isolating CD14+ cells and CD2+ cells from the blood sample; c) determining the gene expression level of 10 or more biomarkers selected from the group consisting of ENTREP1, KIR2DL4, KIF2C, BICC1, ROR2, LOC124904706, DUSP2, LOC122455342, ST6GALNAC2, POU2F3, LOC124908063, DKK3, DKK2, KLRF1, MYO18B, KLRC2, MATN2, FCER1A, GPRC5C, CLCN4, H3-5, LOC105374736, EPB41L4A, KCNJ10, SYNM, MEIS3, FOXD1, IQSEC3, NEBL, PLXNA3, LILRB5, PF4, SIGLEC17P, TPBG, RORB, CSMD1, SCGB3A2, OR1F1, CA2, ITGB3, FST, PPBP, SLC35F3, PPP1R14C, RNF217, ROBO1, LINC01644, LRRC77P, TMEM171, BCAS1, PDE5A, DPYSL4, NAV3, LINC01819, PRUNE2, IGLV3-12, SH3BGRL2, TUBB1, COLEC12, CDK14, KRT1, TMEM255A, SLC44A5, LARGE1, ITGA11, C1QC, WNT5B, DNAH10, COL19A1, XKR9, CELSR1, MEG3, MYOM2, ADAMTS2, TCL1A, ADORA3, ZNF890P, SPIB, DYNLT5, SH3RF2, TRIM58, PTPRB, FZD6, ADGRB3, KREMEN1, SYCE1, OR2W3, NYAP2, UTS2, DOC2B, SORCS2, FSIP2, GRIP1, HLA-DRB6, RAMP3, FCRL1, LOC101929563, LINC01918, CPE, GLIS3, S100B, HBEGF, SFTPB, PTPRG, CFAP95, ORM1, and ANXA9 in the CD14+ cells; d) determining the expression level of the 10 or more selected biomarkers in the CD2+ cells; e) normalizing the expression level of each of the selected biomarkers by determining the log ratio of CD14+ over CD2+ expression levels; f) using the normalized expression level of each of the 10 or more selected biomarkers of step (e) to calculate an active surveillance risk score (ASRS) representing the probability that the patient is harboring aggressive prostate cancer; and g) categorizing the patient into a group selected from very low risk, low/average risk, and high risk based on the patient's ASRS.

In another embodiment, the disclosure provides a method of analyzing a blood sample from a patient with prostate cancer comprising: a) obtaining a blood sample from the patient with prostate cancer; b) isolating CD14+ cells and CD2+ cells from the blood sample; c) determining the gene expression level of 10 or more biomarkers selected from the group consisting of ENTREP1, KIR2DL4, KIF2C, BICC1, ROR2, LOC124904706, DUSP2, LOC122455342, ST6GALNAC2, POU2F3, LOC124908063, DKK3, DKK2, KLRF1, MYO18B, KLRC2, MATN2, FCER1A, GPRC5C, CLCN4, H3-5, LOC105374736, EPB41L4A, KCNJ10, SYNM, MEIS3, FOXD1, IQSEC3, NEBL, PLXNA3, LILRB5, PF4, SIGLEC17P, TPBG, RORB, CSMD1, SCGB3A2, OR1F1, CA2, ITGB3, FST, PPBP, SLC35F3, PPP1R14C, RNF217, ROBO1, LINC01644, LRRC77P, TMEM171, BCAS1, PDE5A, DPYSL4, NAV3, LINC01819, PRUNE2, IGLV3-12, SH3BGRL2, TUBB1, COLEC12, CDK14, KRT1, TMEM255A, SLC44A5, LARGE1, ITGA11, C1QC, WNT5B, DNAH10, COL19A1, XKR9, CELSR1, MEG3, MYOM2, ADAMTS2, TCL1A, ADORA3, ZNF890P, SPIB, DYNLT5, SH3RF2, TRIM58, PTPRB, FZD6, ADGRB3, KREMEN1, SYCE1, OR2W3, NYAP2, UTS2, DOC2B, SORCS2, FSIP2, GRIP1, HLA-DRB6, RAMP3, FCRL1, LOC101929563, LINC01918, CPE, GLIS3, S100B, HBEGF, SFTPB, PTPRG, CFAP95, ORM1, and ANXA9 in the CD14+ cells; d) determining the expression level of the 10 or more selected biomarkers in the CD2+ cells; e) normalizing the expression level of each of the selected biomarkers by determining the log ratio of CD14+ over CD2+ expression levels; and f) using the normalized expression level of each of the 10 or more selected biomarkers of step (e) to calculate an active surveillance risk score (ASRS) representing the probability that the patient is harboring aggressive prostate cancer.

These and other embodiments of the subject disclosure will readily occur to those of skill in the art in view of the disclosure herein.

### BRIEF DESCRIPTION OF DRAWINGS

FIG. 1 shows (A) a calibration plot of immunocyte transcriptomic profile combined with the clinicodemographic risk factors age and PSA density in the independent validation cohort (biopsy-positive patients only). The calibration curve has an estimated intercept and slope of 0.10 (95% CI: 0.03-0.17) and 0.90 (95% CI: 0.74-1.07), respectively, and a Brier score of 0.205 (95% CI: 0.191-0.218); and (B) ROC curves of the immunocyte transcriptomic model, PSA, PSA density (PSAD) and a model combining age and PSAD. The immunocyte transcriptomic model significantly outperformed all other biomarkers (all p<0.01).

FIG. 2 shows (A) Contribution of the individual components to the full immunocyte transcriptomic model, with the RNA signature accounting for 41%. (B) A breakdown of the weights of the genes included in the immunocyte transcriptomic part of the signature, with positive signal indicating increased expression in CD14 relative to CD2, and vice versa.

FIG. 3 shows active surveillance risk score (ASRS) categorization of prostate cancer patients into very low, low/average, and high risk prostate cancer (PCa) categories. Negative predictive value (NPV) and positive predictive value (PPV) were determined based on an assumed prevalence of clinically significant PCa (csPCa) in an active surveillance (AS) population of 25%. Performance parameters were based on the results obtained in the biopsy-positive, independent validation cohort.

FIG. 4 shows clinicodemographic characteristics of the entire training and validation cohorts (including biopsy-negative subjects), and a comparison between the individual parameters. This training set was used to build an immunocyte transcriptomic model of the disclosure.

FIG. 5 shows clinicodemographic characteristics of the biopsy-positive subsets of the training and validation cohorts, and a comparison between the individual parameters. This biopsy-positive subset, serving as a surrogate for an AS population was used to validate the immunocyte transcriptomic model.

FIG. 6 lists biomarker genes useful in the compositions, methods, and systems of the disclosure.

FIG. 7 provides a decision curve analysis on the validation set (biopsy-positive only) depicting the net benefit of the model combining the immunocyte transcriptomic profile with PSA density and age over the model including these clinical risk factors only. For reference, the situation where all men undergo radical treatment for csPCa (solid, sloped line), and the situation where no men undergo further treatment (solid, horizontal line) are also depicted.

### DETAILED DESCRIPTION

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present disclosure. Indeed, the present disclosure is in no way limited to the methods and materials described herein. Terms used in the singular will also include the plural and vice versa. For purposes of the disclosure, the following terms are defined below.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +/−20% or +/−10%, more preferably +1-5%, even more preferably +/−1%, and still more preferably +/−0.1% from the specified value, as such variations are appropriate to perform the disclosed methods.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The terms “tumor” and “lesion” as used herein, refer to all neoplastic cell growth and proliferation, whether malignant or benign, and all pre-cancerous and cancerous cells and tissues. Those skilled in the art will realize that a tumor tissue sample may comprise multiple biological elements, such as one or more cancer cells, partial or fragmented cells, tumors in various stages, surrounding histologically normal-appearing tissue, and/or macro or micro-dissected tissue.

The terms “cancer” and “cancerous” refer to or describe the physiological condition in mammals that is typically characterized by unregulated cell growth. Examples of cancer in the present disclosure include cancer of the urogenital tract, such as prostate cancer.

As used herein, the term “prostate cancer” is used in the broadest sense and refers to all stages and all forms of cancer arising from the tissue of the prostate gland.

Staging of the cancer assists a physician in assessing how far the disease has progressed and to plan a treatment for the patient. Staging may be done clinically (clinical staging) by physical examination, blood tests, or response to radiation therapy, and/or pathologically (pathologic staging) based on surgery, such as radical prostatectomy. According to the tumor, node, metastasis (TNM) staging system of the American Joint Committee on Cancer (AJCC), AJCC Cancer Staging Manual (7th Ed., 2010), the various stages of prostate cancer are defined as follows: Tumor: T1: clinically inapparent tumor not palpable or visible by imaging, T1a: tumor incidental histological finding in 5% or less of tissue resected, T1b: tumor incidental histological finding in more than 5% of tissue resected, T1c: tumor identified by needle biopsy; T2: tumor confined within prostate, T2a: tumor involves one half of one lobe or less, T2b: tumor involves more than half of one lobe, but not both lobes, T2c: tumor involves both lobes; T3: tumor extends through the prostatic capsule, T3a: extracapsular extension (unilateral or bilateral), T3b: tumor invades seminal vesicle(s); T4: tumor is fixed or invades adjacent structures other than seminal vesicles (bladder neck, external sphincter, rectum, levator muscles, or pelvic wall). Generally, a clinical T (cT) stage is T1 or T2 and pathologic T (pT) stage is T2 or higher. Node: NO: no regional lymph node metastasis; N1: metastasis in regional lymph nodes. Metastasis: M0: no distant metastasis; M1: distant metastasis present.

The Gleason Grading system is used to help evaluate the prognosis of men with prostate cancer. Together with other parameters, it is incorporated into a strategy of prostate cancer staging, which predicts prognosis and helps guide therapy. A Gleason “score” or “grade” is given to prostate cancer based upon its microscopic appearance. Tumors with a low Gleason score typically grow slowly enough that they may not pose a significant threat to the patients in their lifetimes. These patients are monitored (“watchful waiting” or “active surveillance”) over time. Cancers with a higher Gleason score are more aggressive and have a worse prognosis, and these patients are generally treated with surgery (e.g., radical prostatectomy) and, in some cases, therapy (e.g., radiation, hormone, ultrasound, chemotherapy). Gleason scores (or sums) comprise grades of the two most common tumor patterns. These patterns are referred to as Gleason patterns 1-5, with pattern 1 being the most well-differentiated. Most have a mixture of patterns. To obtain a Gleason score or grade, the dominant pattern is added to the second most prevalent pattern to obtain a number between 2 and 10. The Gleason Grades include: G1: well differentiated (slight anaplasia) (Gleason 2-4); G2: moderately differentiated (moderate anaplasia) (Gleason 5-6); G3-4: poorly differentiated/undifferentiated (marked anaplasia) (Gleason 7-10).

Stage groupings: Stage I: T1a N0 M0 G1; Stage II: (T1a N0 M0 G2-4) or (T1b, c, T1, T2, N0 M0 Any G); Stage III: T3 N0 M0 Any G; Stage IV: (T4 N0 M0 Any G) or (Any T N1 M0 Any G) or (Any T Any N M1 Any G).

As described in detail herein, the term “active surveillance” refers to closely monitoring a patient's condition without giving or providing treatment until symptoms appear or change. For example, in the context of prostate cancer, active surveillance is utilized to observe, rather than treat (e.g., with surgery, radiation, chemotherapy, and/or subject to unnecessary invasive techniques (e.g., biopsies) and/or treatments) patients with indolent disease.

The term active surveillance risk score (ASRS) refers to a patient-specific, quantitative score that provides information for determining whether or not a patient should be provided and/or administered treatment for prostate cancer. The disclosure provides that a prostate cancer patient with an ASRS of very low risk or low/average risk is a patient that will benefit from (e.g., should enter/begin, remain on, and/or continue) active surveillance. The disclosure also provides that a prostate cancer patient with an ASRS of high risk is a patient that will not benefit from active surveillance (e.g., the patient should not be placed on or continue, and/or should stop) active surveillance (e.g., the patient should receive active intervention for the disease). Compositions, methods, and systems for determining a patient's ASRS are described in detail herein. The terms “subject” and “patient” are used interchangeably herein (e.g., “a subject with prostate cancer” is used interchangeably with “a patient with prostate cancer”).

The terms “upgrade” and “upgrading” in the context of prostate cancer refers to observing certain patterns and/or quantifiable information of disease (e.g., ASRS), and upon the assessment of a specific change in the pattern, a low-risk disease pattern may be “upgraded” to intermediate or higher risk (grade).

As used herein, the term “surgery” applies to surgical methods for removal of cancerous tissue, including pelvic lymphadenectomy, radical prostatectomy, transurethral resection of the prostate (TURP), excision, dissection, and tumor biopsy/removal.

The terms “sample” or “biological sample” or “component” as used herein, refers to a sample of biological fluid, tissue, or cells, in a healthy and/or pathological state obtained from a subject. Such samples include, but are not limited to, blood, bronchial lavage fluid, sputum, saliva, urine, amniotic fluid, lymph fluid, tissue or fine needle biopsy samples, peritoneal fluid, cerebrospinal fluid, nipple aspirates, and includes supernatant from cell lysates, lysed cells, cellular extracts, and nuclear extracts. The terms “sample” or “biological sample” or “component” encompass samples that may comprise protein or nucleic acid material shed from tumor cells in vivo including bone marrow, blood, plasma, serum, and the like.

The term “prognosis” is used herein to refer to the likelihood that a cancer patient will have a cancer-attributable death or progression, including recurrence, metastatic spread, and drug resistance, of a neoplastic disease, such as prostate cancer. For example, a “good prognosis” would include long term survival without recurrence and a “bad prognosis” would include cancer recurrence. Prognosis and clinical outcome may be assessed using any endpoint indicating a quantifiable parameter of the patient, including, without limitation, (1) tumor growth (e.g., inhibition, slowing down and/or complete growth arrest would be an example of a positive clinical outcome/prognosis; (2) number of tumor cells; (3) tumor size; (4) tumor cell infiltration into adjacent peripheral organs and/or tissues; (5) metastasis; (6) anti-tumor immune response; (7) one or more symptoms associated with the tumor; (8) duration of survival following treatment; and/or (9) mortality at a given point of time following treatment. Clinical outcome can also be considered in the context of an individual's outcome relative to an outcome of a population of patients having a comparable clinical diagnosis, and can be assessed using various endpoints such as an increase in the duration of Recurrence-Free Interval (RFI), an increase in survival time (Overall Survival (OS)) or prostate cancer-specific survival time (Prostate Cancer-Specific Survival (PCSS)) in a population, no upstaging or upgrading in tumor stage, Gleason grade, or via a patient's active surveillance risk score (ASRS) defined herein. Thus, an ASRS may be obtained from a patient at any number of timepoints (e.g., at annual check-up, prior to treatment, post treatment (e.g., to monitor residual disease)), or in combination with other tests and/or procedures (e.g., for assessing and/or monitoring prostate cancer (e.g., biopsy, magnetic resonance imaging (MRI), Gleason grading, etc.), genetic testing (e.g., in a patient with a familial history of cancer)).

The term “risk classification” means a grouping of subjects by the level of risk (or likelihood) that the subject will experience a particular negative clinical outcome. A subject may be classified into a risk group or classified at a level of risk based on the methods of the present disclosure, e.g., very low risk, low/average risk, or high risk. A “risk group” is a group of subjects or individuals with a similar level of risk for a particular clinical outcome.

The term “recurrence” is used herein to refer to local or distant recurrence (i.e., metastasis) of cancer. For example, prostate cancer can recur locally in the tissue next to the prostate or in the seminal vesicles. The cancer may also affect the surrounding lymph nodes in the pelvis or lymph nodes outside this area. Prostate cancer can also spread to tissues next to the prostate, such as pelvic muscles, bones, or other organs. Recurrence can be determined by clinical recurrence detected by, for example, imaging study or biopsy, or biochemical recurrence detected by, for example, an active surveillance risk score of high risk.

As used herein, the terms “biomarker” or “marker” or “genomic marker” or “genomic covariate” or “biological marker” refer to an analyte (e.g., a nucleic acid, DNA, RNA, peptide, protein, or metabolite) that can be objectively measured and evaluated as an indicator for a biological process. In some embodiments, a marker is differentially detectable in phagocytes and is indicative of the presence or absence of prostate cancer. An analyte is differentially detectable if it can be distinguished quantitatively or qualitatively in phagocytes compared to a control, e.g., a normal or healthy control or non-phagocytic cells.

As used herein, the term “expression level” as applied to a marker or gene refers to the level of gene expression (e.g., how many copies or transcripts are generated) and may also refer to the level of gene product (e.g., protein, RNA, etc.).

The term “gene product” or “expression product” are used herein to refer to the RNA (ribonucleic acid) transcription products (transcripts) of the gene, including mRNA, and the polypeptide translation products of such RNA transcripts. A gene product can be, for example, an unspliced RNA, an mRNA, a splice variant mRNA, a microRNA, a fragmented RNA, a polypeptide, a post-translationally modified polypeptide, a splice variant polypeptide, etc.

The term “RNA transcript” as used herein refers to the RNA transcription products of a gene, including, for example, mRNA, an unspliced RNA, a splice variant mRNA, a microRNA, and a fragmented RNA.

Unless indicated otherwise, each gene name used herein corresponds to the Official Symbol assigned to the gene and provided by Entrez Gene as of the filing date of this application.

The term “microarray” refers to an ordered arrangement of hybridizable array elements, e.g. oligonucleotide or polynucleotide probes, on a substrate.

The term “polynucleotide” generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons, are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNArDNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The “area under curve” or “AUC” refers to area under a ROC curve. AUC under a ROC curve is a measure of accuracy. An AUC of 1 represents a perfect test, whereas an AUC of 0.5 represents an insignificant test. A preferred AUC may be at least approximately 0.700, at least approximately 0.750, at least approximately 0.800, at least approximately 0.850, at least approximately 0.900, at least approximately 0.910, at least approximately 0.920, at least approximately 0.930, at least approximately 0.940, at least approximately 0.950, at least approximately 0.960, at least approximately 0.970, at least approximately 0.980, at least approximately 0.990, at least approximately 0.995, at least approximately 0.990, at least approximately 0.850, at least approximately 0.800, at least approximately 0.750, at least approximately 0.700, at least approximately 0.650, or at least approximately 0.600.

“Isolated polynucleotide” as used herein may mean a polynucleotide (e.g., of genomic, cDNA, or synthetic origin, or a combination thereof) that, by virtue of its origin, the isolated polynucleotide is not associated with all or a portion of a polynucleotide with which the “isolated polynucleotide” is found in nature; is operably linked to a polynucleotide that it is not linked to in nature; or does not occur in nature as part of a larger sequence.

A “receiver operating characteristic” curve or “ROC” curve refers to a graphical plot that illustrates the performance of a binary classifier system as its discrimination threshold is varied. For example, an ROC curve can be a plot of the true positive rate against the false positive rate for the different possible cutoff points of a diagnostic test. It is created by plotting the fraction of true positives out of the positives (TPR=true positive rate) vs. the fraction of false positives out of the negatives (FPR=false positive rate), at various threshold settings. TPR is also known as sensitivity, and FPR is one minus the specificity or true negative rate. The ROC curve demonstrates the tradeoff between sensitivity and specificity (any increase in sensitivity will be accompanied by a decrease in specificity); the closer the curve follows the left-hand border and then the top border of the ROC space, the more accurate the test; the closer the curve comes to the 45-degree diagonal of the ROC space, the less accurate the test; the slope of the tangent line at a cutoff point gives the likelihood ratio (LR) for that value of the test; and the area under the curve is a measure of test accuracy.

As used herein, the term “characterizing cancer in a subject” refers to the identification of one or more properties of a cancer sample in a subject, including but not limited to, the presence of benign, pre-cancerous or cancerous tissue and the stage and/or aggressiveness of the cancer. In one non-limiting example, compositions and methods of the disclosure are utilized to characterize cancer in a subject (e.g., to identify the aggressiveness or indolence of prostate cancer) in a subject.

“Statistically significant” as used herein refers to the likelihood that a relationship between two or more variables is caused by something other than random chance. Statistical hypothesis testing is used to determine whether the result of a data set is statistically significant. In statistical hypothesis testing, a statistical significant result is attained whenever the observed p-value of a test statistic is less than the significance level defined of the study. The p-value is the probability of obtaining results at least as extreme as those observed, given that the null hypothesis is true. Examples of statistical hypothesis analysis include Wilcoxon signed-rank test, t-test, Chi-Square or Fisher's exact test. “Significant” as used herein refers to a change that has not been determined to be statistically significant (e.g., it may not have been subject to statistical hypothesis testing).

As used herein, “treating” prostate cancer refers to taking active steps in an effort to obtain beneficial or desired results, including clinical results. Beneficial or desired clinical results include, but are not limited to, alleviation or amelioration of one or more symptoms associated with diseases or conditions.

The terms “correlated” and “associated” are used interchangeably herein to refer to the association between two measurements (or measured entities). The disclosure provides genes or gene subsets, the expression levels of which are utilized to generate an active surveillance risk score (e.g., that is associated with clinical outcome). For example, the increased or decreased expression level of a gene may be positively correlated (positively associated) with a good or positive clinical outcome. Such a positive correlation may be demonstrated statistically in various ways, e.g. by a cancer recurrence hazard ratio less than one or by a cancer upgrading or upstaging odds ratio of less than one. In another example, the increased or decreased expression level of a gene may be negatively correlated (negatively associated) with a good or positive clinical outcome. In that case, for example, the patient may experience a cancer recurrence or upgrading/upstaging of the cancer. “Correlation” is also used herein to refer to the strength of association between the expression levels of two or more different genes, such that the expression level of a first gene can be substituted with an expression level of a second gene in a given algorithm if their expression levels are highly correlated. Such “correlated expression” of two or more genes that are substitutable in an algorithm are usually gene expression levels that are positively correlated with one another, e.g., if increased or decreased expression of a first gene is positively correlated with an outcome (e.g., increased likelihood of good clinical outcome), then the second gene that is co-expressed and exhibits correlated expression with the first gene is also positively correlated with the same outcome.

A “computer-based system” refers to a system of hardware, software, and data storage medium used to analyze information. The minimum hardware of a patient computer-based system comprises a central processing unit (CPU), and hardware for data input, data output (e.g., display), and data storage. An ordinarily skilled artisan can readily appreciate that any currently available computer-based systems and/or components thereof are suitable for use in connection with the methods of the present disclosure. The data storage medium may comprise any manufacture comprising a recording of the present information as described above, or a memory access device that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” or “computing means” references any hardware and/or software combination that will perform the functions required of it. For example, a suitable processor may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station

As used herein, “administering” or “administration of” a compound or an agent to a subject can be carried out using one of a variety of methods known to those skilled in the art. For example, a compound or an agent can be administered, intravenously, arterially, intradermally, intramuscularly, intraperitoneally, intravenously, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow, or controlled release of the compound or agent. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. In some embodiments, a compound or an agent is administered orally, e.g., to a subject by ingestion, or intravenously, e.g., to a subject by injection. In some embodiments, the orally administered compound or agent is in an extended release or slow release formulation or administered using a device for such slow or extended release.

The present disclosure provides tools for assessing and managing patients with prostate cancer and in particular those patients with low-risk disease. As disclosed herein, the disclosure provides compositions, methods and systems that utilize a single blood draw from a prostate cancer patient that identifies and distinguishes aggressive prostate cancer (e.g., defined by NCCN unfavorable intermediate and higher risk) from very low risk and low/average risk prostate cancer. The disclosure provides a significantly improved ability to identify those patients that should not be on active surveillance (AS) versus those that benefit from AS without compromising outcome. The compositions, methods, and systems of this disclosure revolutionize the way active surveillance is conducted and ease the burden of numerous repeat biopsies to screen for patients that are erroneously put on an active surveillance pathway (e.g., due to biopsy error).

For a variety of reasons, many men with prostate cancer that are on AS with low or intermediate risk disease forgo the necessary repeat surveillance biopsies needed to identify potentially higher risk PCa. In addition, men with PCa that have not yet been diagnosed with PCa avoid tests and procedures that otherwise would have identified the PCa. The disclosure provides compositions, methods, and systems that utilize a blood-based immunocyte transcriptomic signature model to identify men harboring occult aggressive PCa. As detailed herein, the blood-based immunocyte transcriptomic signature model was validated on a biopsy-positive population to identify men who should not be on AS and confirm those men with indolent disease who can safely remain on AS.

The disclosure provides compositions, methods, and systems useful for the discovery and characterization of prostate cancer (e.g., prostate cancer signatures). In particular, compositions, methods and systems disclosed are useful for generating differential transcriptomic profiles (e.g., of CD14+ and/or CD2+ cell populations) that are associated with and that can predict adverse pathologic features of prostate cancer, and that find use in the identification, prognosis, treatment and/or management of prostate cancer patients (e.g., via generation of a patient-specific active surveillance risk score).

In some embodiments, the blood-based immunocyte transcriptomic signature model uses subtraction-normalized immunocyte transcriptomic profiles to risk stratify men with PCa as candidates for AS (See Example 1). The model was validated in an independent cohort (Example 1). Briefly, and as described in Example 1, men were eligible for enrollment in the study if they were determined by their physician to have a risk profile that warranted prostate biopsy. Both training (n=1017) and validation cohort (n=1198) populations had blood samples drawn coincident to their prostate biopsy. Purified CD2 and CD14 immune cells were obtained from peripheral blood mononuclear cells and RNA was extracted and sequenced. To avoid overfitting and unnecessary complexity, a regularized regression model was built on the training cohort to prognose PCa aggressiveness based on the National Comprehensive Cancer Network (NCCN) prostate cancer guidelines. The model was then independently validated in an independent cohort of biopsy-positive men only, using NCCN unfavorable intermediate risk and worse as an aggressiveness outcome, identifying patients that were not appropriate for AS. As described herein, in some embodiments, a model for the AS setting was obtained by combining an immunocyte transcriptomic profile based on two cell types (phagocyte cells (e.g., CD14+ cells) and non-phagocyte cells (e.g., CD2+ cells)) with prostate-specific antigen (PSA) density (PSAD), and age, reaching an AUC of 0.73 (95% confidence interval (CI): 0.69-0.77). The model significantly outperformed (p<0.001) PSA density as a biomarker, which has an AUC of 0.69 (95% CI: 0.65-0.73). The model yields an individualized patient risk score with 90% negative predictive value (NPV) and 50% positive predictive value (PPV). Thus, the disclosure, provides compositions, methods, and systems utilizing the a blood-based immunocyte transcriptomic signature model for risk stratification of individual PCa patients to provide data for personalized decision making with respect to the decision to enter, continue, or stop an AS pathway.

Immune cells from the adaptive (T- and B-lymphocytes) and innate (myeloid) immune systems play a prominent role in the initiation, progression, metastasis, and treatments of prostate cancer (32). The immune characteristics of the PCa tumor microenvironment can be a useful tool for determining response to immunotherapy (33). The interplay between innate and adaptive immunity participates as a positive and negative regulator of the adaptive immunosurveillance. The presence of tumor cells may skew leukocytes expression profiles towards an immunosuppressive state and degrade the phenotypic plasticity of the immune compartment and lead to disease progression (34). Innate immune checkpoints can interfere with the phagocytic cell detection and clearance of tumor cells and thereby suppress innate sensing, leading to immune escape of tumor (35).

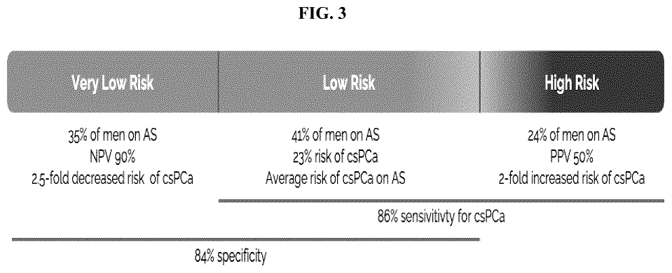

The immunocyte transcriptomic model that was generated during development of embodiments of the disclosure allows for significantly improved clinical management of PCa patients on AS using non-invasive and/or minimally invasive sampling (e.g., a single blood draw). In some embodiments, this non- or minimially-invasive sampling avoids the use, or minimize the use, of surveillance biopsies and mpMRI. As detailed herein, the product of this model was a quantitative risk score (active surveillance risk score (ASRS)) allowing for an objective interpretation of an individual patient's risk of harboring aggressive PCa (e.g., and subsequently, use of the ASRS in the determination of whether or not the patient should enter or remain on AS, or, if the patient should not enter or stop AS (e.g., and instead receive active treatment for PCa)). Although the disclosure is not limited to any particular reference point, in some embodiments, two reference points were identified and utilized on a continuous risk scale that made possible the identification of approximately one-third of men with low-risk disease (90% NPV) and another third with likely high-risk disease (50% PPV), respectively (see, e.g., Example 1 and FIG. 3). In some embodiments, risk score categories were generated for categorizing men in very low, low/average, and high risk PCa (NPV and PPV were determined based on an assumed prevalence of clinically significant PCa (csPCa) in an AS population of 25% (see Example 1 and FIG. 3).

While an understanding of the mechanism is not needed to practice the present disclosure and wherein the present disclosure is not limited to any specific mechanism, in some embodiments, the significant increase in performance of the compositions, methods, and systems of the disclosure that utilize the immunocyte transcriptomic signature model over the conventional prostate cancer tests (e.g., biopsy, mpMRI, etc.) and markers (PSA) is based on the fact that cancer and immune pathways are associated with genes in this white blood cell-based signature model.

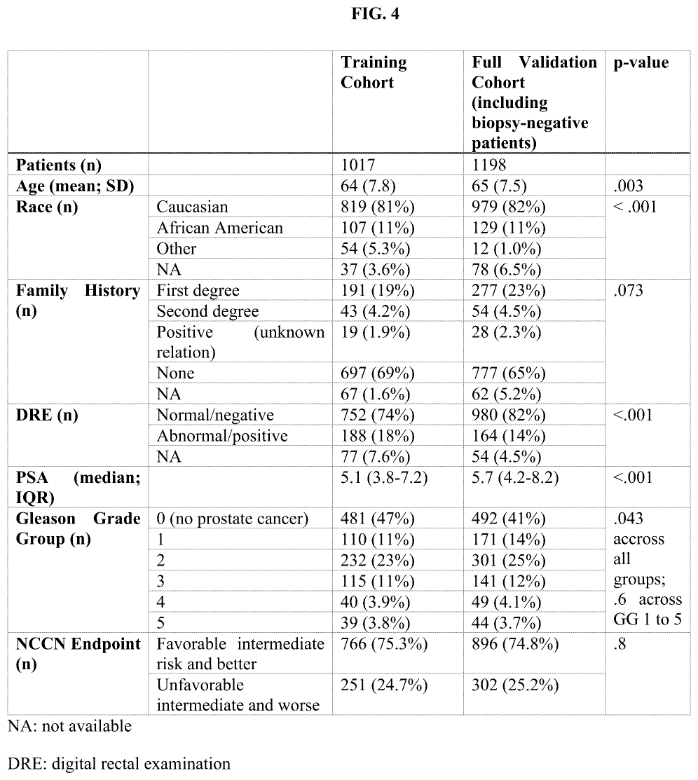

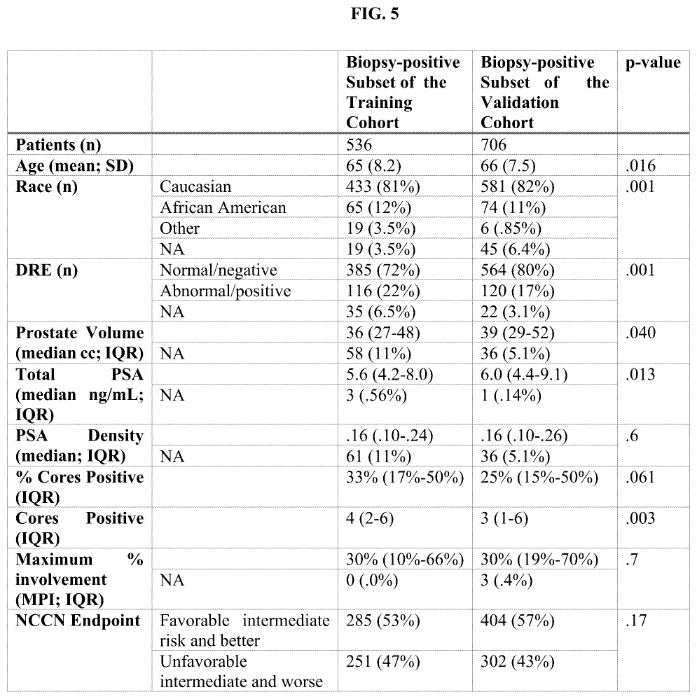

The patients in the validation cohort studied during the development of embodiments of the disclosure had a median follow-up of 25 months. The use of progression to NCCN unfavorable intermediate clinicopathologic features, or worse, as endpoint was the best short-term substitute forgoing analyses requiring clinical endpoints such as disease-specific mortality and metastasis, which are difficult to obtain and understand given the current aggressive treatment protocols. Moreover, upgrading by biopsy is a clinically actionable finding, thus a clinically important endpoint. Disease progression to or beyond NCCN unfavorable intermediate is a good and safe observation in a cohort undergoing delayed intent-to-treat monitoring, i.e. AS. A possible limitation of the disclosure is the missingness of data, in particular PSA density. This is most often due to missing prostate volume. However, in some embodiments, missing data can be imputed so that the parameters age and PSA density can be included. While a single imputation method has limitations, more sophisticated imputation methods that were tested did not improve model performance. While an understanding of a mechanism is not needed to practice the disclosure, and the disclosure is not limited to any particular mechanism, the failure of more sophisticated models to perform better may be because missing data is largely due to missing prostate volume, which, as disclosed here, has no direct correlation with other clinicodemographic (risk) parameters in a cohort of men with PCa. The reduced missingness (less than 10%) in the validation set limited the potential for bias (See FIGS. 4 and 5).

Contemporary studies on large cohorts indicate that AS is a safe strategy over longer follow-up for appropriately identified/selected patients with favorable pathology when following a well-defined monitoring plan (36). The recent introduction of mpMRI into the diagnostic PCa care pathway with improved biopsy targeting accuracy has led to the widening of both the AS inclusion and follow-up criteria, which can be accomplished without undesirably exposing patients to the increased risk of overtreatment (37). While useful, the PRECISE criteria for serial mpMRI of the prostate during AS cannot replace initial or follow-up biopsies (38-40). In some embodiments, mpMRI is utilized in an AS pathways together with the compositions, methods, and/or systems of the disclosure that utilize the immunocyte transcriptomic signature model disclosed herein.

In some embodiments, compositions, methods, and/or systems of the disclosure that utilize the immunocyte transcriptomic signature model are used for risk stratification of individual patients (e.g., provide data for personalized decision making with respect to enter, continue, or stop an AS pathway). In some embodiments, the high NPV results in more patients avoiding defecting from AS when they truly have indolent disease. In other embodiments, the compositions, methods, and/or systems of the disclosure provide assessment of the risk of a patient harboring aggressive PCa (e.g., ASRS of high risk) that is higher than low or low/intermediate risk (e.g., relative to a population of patients with prostate cancer or to the general population), allowing a patient with a high risk ASRS to move on to definite therapy without the need for additional biopsies.

Thus, in some embodiments, the present disclosure provides compositions, methods, and systems useful for the assessment of prostate cancer patients for active surveillance of the prostate cancer, as well as compositions, methods, and systems for assessing and identifying prostate cancer. The disclosure provides algorithm-based assays comprising subtraction-normalized immunocyte signature profiling from a sample obtained from a prostate cancer patient. Measurement of expression of signature markers identify on an individual basis, via an active surveillance risk score (ASRS), prostate cancer patients that are to enter, continue, or stop an active surveillance pathway.

### Algorithm-Based Methods and Gene Subsets.

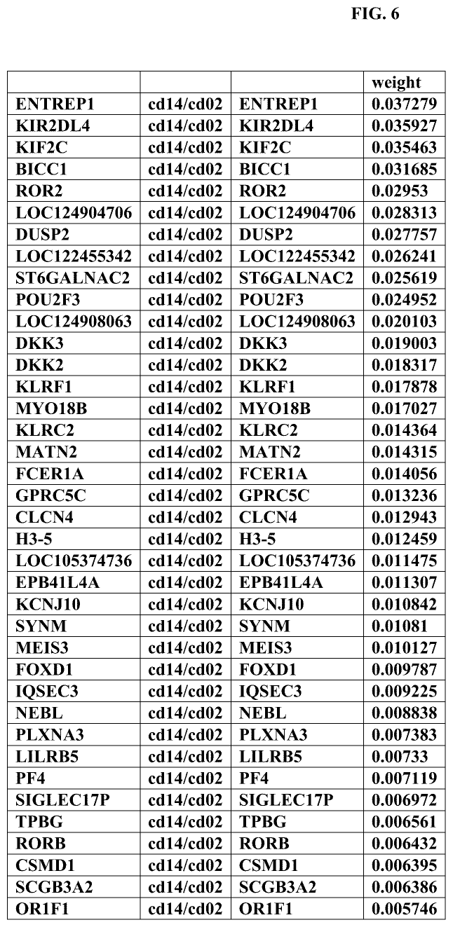

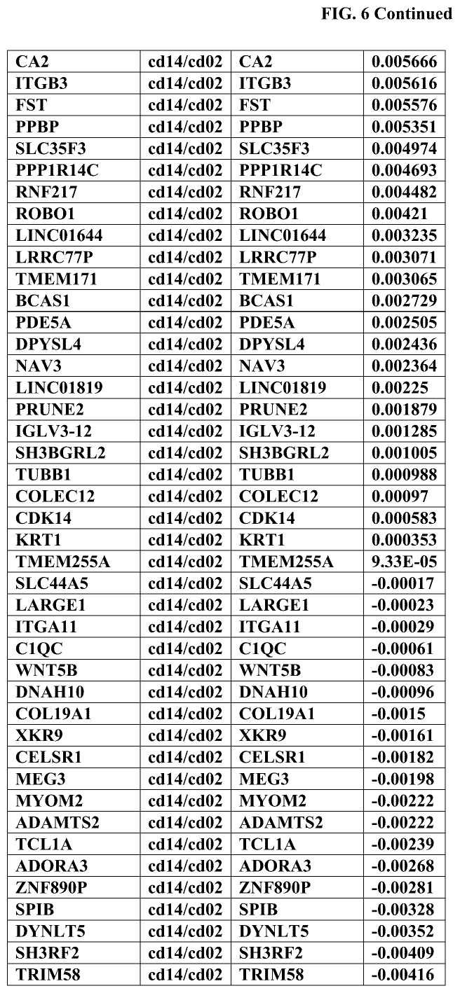

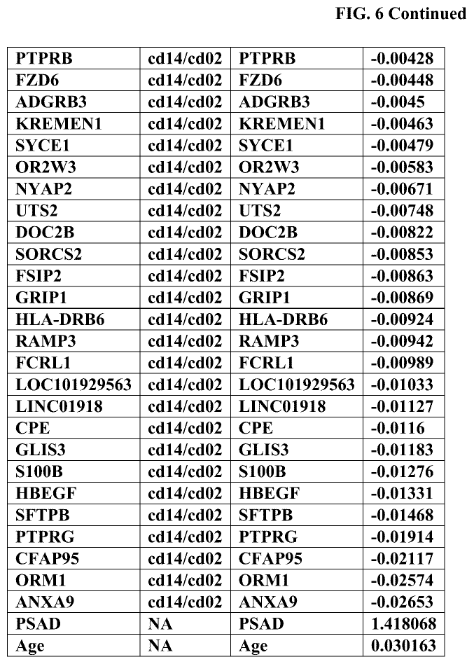

The present disclosure provides, in one embodiment, an algorithm-based molecular diagnostic assay for predicting a clinical outcome for a patient with prostate cancer. The expression level of a plurality of genes (e.g., from genes identified in FIG. 6) may be used alone or arranged into functional gene subsets to calculate a quantitative score (ASRS) that can be used to predict the likelihood of a clinical outcome (e.g., indolent or aggressive cancer). The algorithm-based assay and associated information provided by the practice of the methods of the present disclosure facilitate optimal treatment decision-making in prostate cancer. For example, such a clinical tool enables physicians to identify patients who have a low likelihood of having an aggressive cancer (e.g., ASRS of very low risk or low/average risk) and therefore could be identified as a candidate for active surveillance and/or not a candidate for active treatment (e.g., surgery such as radical prostatectomy, chemotherapy, radiation therapy, or other type of treatment).

As used herein, a “quantitative score” is an arithmetically or mathematically calculated numerical value for aiding in simplifying or disclosing or informing the analysis of more complex quantitative information, such as the correlation of certain expression levels of the disclosed genes or gene subsets to a likelihood of a clinical outcome of a prostate cancer patient. A quantitative score may be determined by the application of a specific algorithm. The algorithm used to calculate the quantitative score in the methods disclosed herein may utilize the expression level values of genes or groups of genes. The grouping of genes may be performed at least in part based on knowledge of the relative contribution of the genes according to physiologic functions or component cellular characteristics, or by assigning a mathematical weighting of the contribution of various expression levels of genes or gene subsets to the quantitative score (e.g., as shown in the examples). The weighting of a gene or plurality of genes representing a physiological process or component cellular characteristic can reflect the contribution of that process or characteristic to the pathology of the cancer and clinical outcome, such as recurrence or upgrading/upstaging of the cancer. The present disclosure provides a number of algorithms for calculating the quantitative scores, for example, as set forth in the examples.

In one embodiment, the present disclosure provides a method of predicting and/or determining whether a patient with prostate cancer should enter, continue, and/or stop active surveillance comprising determination of a level of a plurality of RNA transcripts, or an expression product thereof, in a biological sample obtained from the patient, wherein the RNA transcript, or its expression product, is selected from genes shown in FIG. 6. In some embodiments, the method comprises calculating a quantitative score (e.g., an active surveillance risk score (ASRS)) for the prostate cancer patient by weighting the level of the one or more RNA transcripts or an expression product thereof, by their contribution to a clinical outcome and predicting and/or determining whether the patient with prostate cancer should enter, continue, and/or stop active surveillance based on the quantitative score. In one embodiment, the active surveillance risk score (ASRS) of very low risk or low/average risk may be used to identify a patient with prostate cancer as a candidate for active surveillance. In one embodiment, the one or more RNA transcripts, or an expression product thereof, is selected from ENTREP1, KIR2DL4, KIF2C, BICC1, ROR2, LOC124904706, DUSP2, LOC122455342, ST6GALNAC2, POU2F3, LOC124908063, DKK3, DKK2, KLRF1, MYO18B, KLRC2, MATN2, FCER1A, GPRC5C, CLCN4, H3-5, LOC105374736, EPB41L4A, KCNJ10, SYNM, MEIS3, FOXD1, IQSEC3, NEBL, PLXNA3, LILRB5, PF4, SIGLEC17P, TPBG, RORB, CSMD1, SCGB3A2, OR1F1, CA2, ITGB3, FST, PPBP, SLC35F3, PPP1R14C, RNF217, ROBO1, LINC01644, LRRC77P, TMEM171, BCAS1, PDE5A, DPYSL4, NAV3, LINC01819, PRUNE2, IGLV3-12, SH3BGRL2, TUBB1, COLEC12, CDK14, KRT1, TMEM255A, SLC44A5, LARGE1, ITGA11, C1QC, WNT5B, DNAH10, COL19A1, XKR9, CELSR1, MEG3, MYOM2, ADAMTS2, TCL1A, ADORA3, ZNF890P, SPIB, DYNLT5, SH3RF2, TRIM58, PTPRB, FZD6, ADGRB3, KREMEN1, SYCE1, OR2W3, NYAP2, UTS2, DOC2B, SORCS2, FSIP2, GRIP1, HLA-DRB6, RAMP3, FCRL1, LOC101929563, LINC01918, CPE, GLIS3, S100B, HBEGF, SFTPB, PTPRG, CFAP95, ORM1, and ANXA9, or a combination thereof.

Any one or more combinations of genes may be assayed in the method of the present disclosure. In one embodiment, the present disclosure provides a method for determining whether a patient with prostate cancer should enter, continue, and/or stop active surveillance of the prostate cancer (e.g., based on assessing the likely clinical outcome for the patient with prostate cancer) comprising measuring the level of expression of a plurality of biomarker genes (e.g., 2 or more, 5 or more, 10 or more, 20 or more, 30 or more, 40 or more, 50 or more, 60 or more, 70 or more, 80 or more, 90 or more biomarker genes) comprising ENTREP1, KIR2DL4, KIF2C, BICC1, ROR2, LOC124904706, DUSP2, LOC122455342, ST6GALNAC2, POU2F3, LOC124908063, DKK3, DKK2, KLRF1, MYO18B, KLRC2, MATN2, FCER1A, GPRC5C, CLCN4, H3-5, LOC105374736, EPB41L4A, KCNJ10, SYNM, MEIS3, FOXD1, IQSEC3, NEBL, PLXNA3, LILRB5, PF4, SIGLEC17P, TPBG, RORB, CSMD1, SCGB3A2, OR1F1, CA2, ITGB3, FST, PPBP, SLC35F3, PPP1R14C, RNF217, ROBO1, LINC01644, LRRC77P, TMEM171, BCAS1, PDE5A, DPYSL4, NAV3, LINC01819, PRUNE2, IGLV3-12, SH3BGRL2, TUBB1, COLEC12, CDK14, KRT1, TMEM255A, SLC44A5, LARGE1, ITGA11, C1QC, WNT5B, DNAH10, COL19A1, XKR9, CELSR1, MEG3, MYOM2, ADAMTS2, TCL1A, ADORA3, ZNF890P, SPIB, DYNLT5, SH3RF2, TRIM58, PTPRB, FZD6, ADGRB3, KREMEN1, SYCE1, OR2W3, NYAP2, UTS2, DOC2B, SORCS2, FSIP2, GRIP1, HLA-DRB6, RAMP3, FCRL1, LOC101929563, LINC01918, CPE, GLIS3, S100B, HBEGF, SFTPB, PTPRG, CFAP95, ORM1, and ANXA9, or a combination thereof, in a biological sample obtained from a subject; and calculating an active surveillance risk score (ASRS) based on the level of expression of the plurality of biomarkers. The active surveillance risk score (ASRS) can be used by a physician as an indicator of the probability that the subject with prostate cancer harbors (e.g., currently harbors or will harbor in the future) aggressive prostate cancer. In one embodiment, an active surveillance risk score (ASRS) of very low risk or an ASRS of low/average risk provides a physician with a quantitative measurement of the likelihood that the patient with prostate cancer is a suitable candidate for active surveillance (e.g., that it would be reasonable for the physician to recommend the patient enters or remains on active surveillance (e.g., instead of receiving treatment for prostate cancer)). In some embodiments, an ASRS score of very low risk or an ASRS of low/average risk indicates that the patient with prostate cancer, in consultation with his physician, may want to consider entering active surveillance. In some embodiments, an ASRS score of high risk provides a physician with a quantitative measurement of the likelihood that the patient with prostate cancer is not a suitable candidate for active surveillance (e.g., that the patient instead should receive prostate cancer treatment). In some embodiments, an ASRS score of very low risk or an ASRS of low/average risk indicates that it is desirable for the patient with prostate cancer, in consultation with his physician, to start, remain on, and/or continue active surveillance. In some embodiments, an ASRS score of high risk indicates that it is desirable that the patient with prostate cancer, in consultation with his physician, stops active surveillance. In some embodiments, a prostate cancer patient is classified with an ASRS of very low risk if the patient has a certain probability (e.g., 25 (+/−about 20, about 19, about 18, about 17, about 16, about 15, about 14, about 13, about 12, about 11, about 10, about 9, about 8, about 7, about 6, about 5, about 3, about 3, about 2) %) of harboring aggressive prostate cancer (e.g., determined by the methods disclosed herein (e.g., from a biological sample from a prostate cancer patient determining the normalized expression of 2 or more, 5 or more, 10 or more, 20 or more, 30 or more, 40 or more, 50 or more, 60 or more, 70 or more, 80 or more, 90 or more biomarker genes of FIG. 6 from phagocytic and non-phagocytic cells of the sample using the log ratio of macrophage cell expression level divided by non-macrophage cell expression level to classify with and/or assign an ASRS of very low risk)). In some embodiments, a prostate cancer patient is classified with an ASRS of low/average risk if the patient has a certain probability (e.g., 50 (+/−about 25, about 24, about 23, about 22, about 21, about 20, about 19, about 18, about 17, about 16, about 15, about 14, about 13, about 12, about 11, about 10, about 9, about 8, about 7, about 6, about 5, about 3, about 3, about 2) %) of harboring aggressive prostate cancer (e.g., determined by the methods disclosed herein). In some embodiments, a prostate cancer patient is classified with an ASRS of high risk if the patient has a certain probability (e.g., 75 (+/−about 25, about 24, about 23, about 22, about 21, about 20, about 19, about 18, about 17, about 16, about 15, about 14, about 13, about 12, about 11, about 10, about 9, about 8, about 7, about 6, about 5, about 3, about 3, about 2) %) of harboring aggressive prostate cancer (e.g., determined by the methods disclosed herein).

The disclosure is not limited by the subset of genes/biomarkers utilized in the compositions and methods described herein (e.g., for determining a patient's ASRS). Examples of subsets of biomarkers useful in the compositions and methods of the disclosure include, but are not limited to, ENTREP1, KIR2DL4, KIF2C, BICC1, ROR2, SFTPB, PTPRG, CFAP95, ORM1, and ANXA9; or ENTREP1, KIR2DL4, KIF2C, BICC1, ROR2, LOC124904706, DUSP2, LOC122455342, ST6GALNAC2, POU2F3, LINC01918, CPE, GLIS3, S100B, HBEGF, SFTPB, PTPRG, CFAP95, ORM1, and ANXA9; or ENTREP1, KIR2DL4, KIF2C, BICC1, ROR2, LOC124904706, DUSP2, LOC122455342, ST6GALNAC2, POU2F3, LOC124908063, DKK3, DKK2, KLRF1, MYO18B, GRIP1, HLA-DRB6, RAMP3, FCRL1, LOC101929563, LINC01918, CPE, GLIS3, S100B, HBEGF, SFTPB, PTPRG, CFAP95, ORM1, and ANXA9; or ENTREP1, KIR2DL4, KIF2C, BICC1, ROR2, LOC124904706, DUSP2, LOC122455342, ST6GALNAC2, POU2F3, LOC124908063, DKK3, DKK2, KLRF1, MYO18B, KLRC2, MATN2, FCER1A, GPRC5C, CLCN4, NYAP2, UTS2, DOC2B, SORCS2, FSIP2, GRIP1, HLA-DRB6, RAMP3, FCRL1, LOC101929563, LINC01918, CPE, GLIS3, S100B, HBEGF, SFTPB, PTPRG, CFAP95, ORM1, and ANXA9; or ENTREP1, KIR2DL4, KIF2C, BICC1, ROR2, LOC124904706, DUSP2, LOC122455342, ST6GALNAC2, POU2F3, LOC124908063, DKK3, DKK2, KLRF1, MYO18B, KLRC2, MATN2, FCER1A, GPRC5C, CLCN4, H3-5, LOC105374736, EPB41L4A, KCNJ10, SYNM, FZD6, ADGRB3, KREMEN1, SYCE1, OR2W3, NYAP2, UTS2, DOC2B, SORCS2, FSIP2, GRIP1, HLA-DRB6, RAMP3, FCRL1, LOC101929563, LINC01918, CPE, GLIS3, S100B, HBEGF, SFTPB, PTPRG, CFAP95, ORM1, and ANXA9; or any one of the above subsets combined with one or more of MEIS3, FOXD1, IQSEC3, NEBL, PLXNA3, LILRB5, PF4, SIGLEC17P, TPBG, RORB, CSMD1, SCGB3A2, OR1F1, CA2, ITGB3, FST, PPBP, SLC35F3, PPP1R14C, RNF217, ROBO1, LINC01644, LRRC77P, TMEM171, BCAS1, PDE5A, DPYSL4, NAV3, LINC01819, PRUNE2, IGLV3-12, SH3BGRL2, TUBB1, COLEC12, CDK14, KRT1, TMEM255A, SLC44A5, LARGE1, ITGA11, C1QC, WNT5B, DNAH10, COL19A1, XKR9, CELSR1, MEG3, MYOM2, ADAMTS2, TCL1A, ADORA3, ZNF890P, SPIB, DYNLT5, SH3RF2, TRIM58, PTPRB, FZD6, ADGRB3, KREMEN1, SYCE1, and OR2W3.

In a specific embodiment, a method of the disclosure comprises measuring the expression levels of genes shown in FIG. 6. In a further embodiment, an ASRS is calculated according to the algorithm(s) described in Example 1.

A method of characterizing/classifying a patient with prostate cancer as very low risk, low/average risk, and high risk of the disclosure may be used in a variety of applications and settings. For example, in one embodiment, a method of characterizing/classifying a patient with prostate cancer as very low risk, low/average risk, and high risk of the disclosure is used to identify an aggressive prostate cancer phenotype in a subject. In another embodiment, a method of characterizing/classifying a patient with prostate cancer as very low risk, low/average risk, and high risk of the disclosure is used to monitor and/or characterize prostate cancer disease progression. In still another embodiment, a method of characterizing/classifying a patient with prostate cancer as very low risk, low/average risk, and high risk of the disclosure is used in a method of analyzing a blood sample.

A method of characterizing/classifying (e.g., determining ASRS for) a patient with prostate cancer as very low risk, low/average risk, and high risk of the disclosure can be used in combination with one or more variables including, but not limited to, adjusted life expectancy, disease characteristics, predicted outcomes, and/or patient preferences and can be considered by the patient and the patient's physician (e.g., in a shared decision process (e.g., in order to tailor prostate cancer therapy or active surveillance for the individual patient)).

For example, a method of characterizing/classifying a patient with prostate cancer as very low risk, low/average risk, and high risk of the disclosure can be used to accurately risk stratify patients based on genetic information (e.g., expression of genes/biomarkers disclosed herein) and informs a patient and/or the patient's physician regarding biochemical recurrence, metastatic disease, or prostate-cancer specific mortality. Because existing active surveillance and treatment paradigms are reliant upon risk assessment, the ability to more accurately risk stratify has significant utility in the management of prostate cancer. Thus, in some embodiments, the methods disclosed herein provide clinically actionable information (e.g., either one time or over a period of time) that fits into existing evidence-based or consensus-recommended prostate cancer treatment paradigms. In some embodiments, a method of characterizing/classifying a patient with prostate cancer with an ASRS of very low risk, low/average risk, or high risk of the disclosure is more accurate (e.g., significantly more accurate) than existing NCCN clinical criteria to predict risk of or occurrence of metastasis. In some embodiments, a method of characterizing/classifying a patient with prostate cancer with an ASRS of very low risk, low/average risk, or high risk of the disclosure is used to predict prostate cancer-specific mortality. In some embodiments, a method of characterizing/classifying a patient with prostate cancer with an ASRS of very low risk, low/average risk, or high risk of the disclosure is used to predict risk of or occurrence of metastasis after prostate cancer therapy. In some embodiments, a method of characterizing/classifying a patient with prostate cancer with an ASRS of very low risk, low/average risk, or high risk of the disclosure is used to predict risk of or occurrence of metastasis in men treated with definitive radiation (e.g., external beam radiation therapy and/or brachytherapy with neoadjuvant ADT).

In some embodiments, a method of characterizing/classifying a patient with prostate cancer with an ASRS of very low risk, low/average risk, or high risk of the disclosure provides an individualized assessment and characterization of prostate cancer tumor aggressiveness (e.g., for use by a prostate cancer patient and/or the patient's physician (e.g., in a shared decision process (e.g., in order to tailor prostate cancer therapy or active surveillance for the individual patient))). For example, when a patient and physician review a patient's ASRS provided by the disclosure, in some embodiments, the decision to enter or to stay on active surveillance (e.g., when the patient's ASRS is very low risk or low/average risk), or to exit active surveillance for treatment or to receive treatment instead of surveillance (e.g., when the patient's ASRS is low/average risk or high risk) is determined by a shared decision making process between the patient and the physician that reflects the patient's understanding of the possible benefits and risks and that accounts for the patient's preferences and values.

In some embodiments, a method of characterizing/classifying a patient with prostate cancer with an ASRS of very low risk, low/average risk, or high risk of the disclosure outperforms clinical and pathological risk factors currently used in standard practice (e.g., pre-treatment PSA, clinical stage, Gleason Score/grade group or nomograms) while at the same time being non-invasive (e.g., thereby increasing patient compliance with testing). Thus, the methods of the disclosure provide a physician the ability to determine if a prostate cancer patient is a candidates for active surveillance (e.g., likely to have a good outcome without immediate definitive treatment) versus a prostate cancer patient that is a candidate for treatment (e.g., that would benefit from receiving the oncologic benefits of immediate or intensified treatment modalities).

In some embodiments, a method of characterizing/classifying a patient with prostate cancer into an ASRS group of very low risk, low/average risk, or high risk of the disclosure is utilized in combination with one or more other guidelines/recommendations for prostate cancer care and/or management. The disclosure is not limited by the source of the guidelines for prostate care and/or management. Indeed, any guidelines for care and/or management known in the art may be used in combination with the methods and biomarkers of the disclosure including, but not limited to, those provided by the American Cancer Society (ACS), the National Comprehensive Cancer Network (NCCN), the American Urological Association (AUA)/Society of Urologic Oncology (SUO), the U.S. Preventive Services Task Force (USPSTF), the European Society for Medical Oncology (ESMO), and the European Association of Urology/European Association of Nuclear Medicine/European Society for Radiotherapy and Oncology/European Society of Urogenital Radiology/International Society of Urological Pathology/International Society of Geriatric Oncology (EAU/EANM/ESTRO/ESUR/ISUP/SIOG).

In some embodiments, a prostate cancer patient classified with and ASRS of very low risk or at the lower end of low/average risk (e.g., classified with an ASRS of 45, 44, 43, 42, 41, 40, 39, 38, 37, 36, 35, 34, 33, 32, 31 or lower percent probability of harboring aggressive prostate cancer determined by a method disclosed herein), in consultation with the patient's physician, is identified as patient that would benefit from active surveillance or is to remain on active surveillance. In some embodiments, a prostate cancer patient classified with and ASRS of high risk or at the higher end of low/average risk (e.g., classified with an ASRS of 65, 66, 67, 68, 69 or higher percent probability of harboring aggressive prostate cancer determined by a method disclosed herein), in consultation with the patient's physician, is identified as patient that would benefit from treatment or that is not a candidate for active surveillance or that if on active surveillance should be removed therefrom. As detailed herein, a prostate cancer patient together with the patient's physician may use the patient's ASRS in combination with one or more other guidelines/recommendations for prostate cancer care and/or management in a shared decision making process to determine the course of treatment and/or surveillance of the patient's prostate cancer. The patient may have been on active surveillance (e.g., as a result of utilizing the compositions and methods of this disclosure) in order to avoid unnecessary treatment, or the patient may have just been diagnosed with prostate cancer and then, using the compositions and methods described herein, was classified with an ASRS of high risk. In some embodiments, a method of classifying the ASRS of a patient's prostate cancer is utilized during the time that the patient is receiving treatment for prostate cancer, and, if the patient's ASRS improves (that is, the patient is no longer classified as high risk), then the patient may enter active surveillance of the prostate cancer with treatment of the prostate cancer ending.

A patient receiving treatment for prostate cancer may receive any type of treatment known in the art. Prostate cancer treatment includes, but it not limited to, surgical intervention, external beam radiation therapy (EBRT), abiraterone, abiraterone with dexamethasone, enzalutamide, apalutamide, darolutamide, androgen deprivation therapy (ADT), ADT with abiraterone, apalutamide, or enzalutamide, triplet therapy of ADT with docetaxel and abiraterone or darolutamide, or ADT with external beam radiation therapy (EBRT), chemotherapy, docetaxel, cabazitaxel, cabazitaxel plus carboplatin, sipuleucel-T, pembrolizumab, mitoxantrone, olaparib, olaparib plus abiraterone, rucaparib, talazoparib, niraparib, immunotherapy, radiopharmaceuticals (e.g., lutetium Lu 177, radium-223).

In some embodiments, the disclosure provides utilizing a method of the disclosure to determine a patient's ASRS, followed by treatment of the patient (e.g., for a prostate cancer patient classified with and ASRS of high risk or at the higher end of low/average risk (e.g., classified with an ASRS of 65, 66, 67, 68, 69 or higher percent probability of harboring aggressive prostate cancer)), followed by utilizing a method of the disclosure to determine the patient's ASRS post treatment. In some embodiments, when the patient's ASRS post treatment is determined to be very low risk or at the lower end of low/average risk (e.g., classified with an ASRS of 45, 44, 43, 42, 41, 40, 39, 38, 37, 36, 35, 34, 33, 32, 31 or lower percent probability of harboring aggressive prostate cancer), the ASRS is used (e.g., by the patient and/or the patient's physician in a shared decision process) to determine that the patient should commence active surveillance of the patient's prostate cancer. In some embodiments, periodically (e.g., every 3, 6, 12, 18, 24, 30, 36, 48 or more months or any timeframe therebetween) determining a patient's ASRS using methods disclosed herein is performed and the ASRS utilized, alone or in combination with one or more other guidelines/recommendations for prostate cancer care and/or management, in a shared decision making process to determine the course of treatment and/or surveillance of the patient's prostate cancer.

Various technological approaches for determination of expression levels of the genes/biomarkers utilized in the compositions and methods for assessing prostate cancer (e.g., for determining a patient's ASRS) are set forth herein, including, without limitation, RT-PCR, microarrays, high-throughput sequencing, serial analysis of gene expression (SAGE) and Digital Gene Expression (DGE). In particular aspects, the expression level of each gene may be determined in relation to various features of the expression products of the gene including exons, introns, protein epitopes and protein activity.

The expression product that is assayed can be, for example, RNA or a polypeptide. The expression product may be fragmented. For example, the assay may use primers that are complementary to target sequences of an expression product and could thus measure full transcripts as well as those fragmented expression products containing the target sequence.

The RNA expression product may be assayed directly or by detection of a cDNA product resulting from a PCR-based amplification method, e.g., quantitative reverse transcription polymerase chain reaction (qRT-PCR). (See e.g., U.S. Pat. No. 7,587,279). Polypeptide expression product may be assayed using immunohistochemistry (IHC) by proteomics techniques. Further, both RNA and polypeptide expression products may also be assayed using microarrays.

Methods of measuring the expression levels of a gene product (e.g., gene expression profiling) include methods based on hybridization analysis of polynucleotides, methods based on sequencing of polynucleotides, and proteomics-based methods. Exemplary methods known in the art for the quantification of RNA expression in a sample include northern blotting and in situ hybridization (Parker & Barnes, Methods in Molecular Biology 106:247-283 (1999)); RNAse protection assays (Hod, Biotechniques 13:852-854 (1992)); and PCR-based methods, such as reverse transcription PCR (RT-PCR) (Weis et al., Trends in Genetics 8:263-264 (1992)). Antibodies may be employed that can recognize sequence-specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS). Other methods known in the art may be used.

Reverse transcription PCR (RT-PCR) may be used. Typically, mRNA is isolated from a test sample. The starting material is typically total RNA isolated from cells (e.g., from blood containing CD14+ and CD+2 cells). General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995). In particular, RNA isolation can be performed using a purification kit, buffer set and protease from commercial manufacturers, such as Qiagen or Promega, according to the manufacturer's instructions. For example, total RNA from cells in culture can be isolated using Qiagen RNeasy mini-columns. Other commercially available RNA isolation kits include MasterPure™ Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test).

The sample containing the RNA is then subjected to reverse transcription to produce cDNA from the RNA template, followed by exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, CA, USA), following the manufacturer's instructions. The derived cDNA can then be used as a template in the subsequent PCR reaction.

PCR-based methods use a thermostable DNA-dependent DNA polymerase, such as a Taq DNA polymerase. For example, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction product. A third oligonucleotide, or probe, can be designed to facilitate detection of a nucleotide sequence of the amplicon located between the hybridization sites the two PCR primers. The probe can be detectably labeled, e.g., with a reporter dye, and can further be provided with both a fluorescent dye, and a quencher fluorescent dye, as in a Tagman® probe configuration. Where a Tagman® probe is used, during the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, high-throughput platforms such as the ABI PRISM 7700 Sequence Detection System® (Perkin-Elmer-Applied Biosystems, Foster City, Calif., USA), or Lightcycler (Roche Molecular Biochemicals, Mannheim, Germany). In a preferred embodiment, the procedure is run on a LightCycler® 480 (Roche Diagnostics) real-time PCR system, which is a microwell plate-based cycler platform.

5′-Nuclease assay data are commonly initially expressed as a threshold cycle (“Ct”). Fluorescence values are recorded during every cycle and represent the amount of product amplified to that point in the amplification reaction. The threshold cycle (Ct) is generally described as the point when the fluorescent signal is first recorded as statistically significant. Alternatively, data may be expressed as a crossing point (“Cp”). The Cp value is calculated by determining the second derivatives of entire qPCR amplification curves and their maximum value. The Cp value represents the cycle at which the increase of fluorescence is highest and where the logarithmic phase of a PCR begins.

To minimize errors and the effect of sample-to-sample variation, RT-PCR can be performed using an internal standard. Gene expression measurements can be normalized relative to the mean of one or more (e.g., 2, 3, 4, 5, or more) reference genes.

Real time PCR is compatible both with quantitative competitive PCR, where an internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

The steps of a representative protocol for use in the methods of the present disclosure use fixed, paraffin-embedded tissues as the RNA source. For example, mRNA isolation, purification, primer extension and amplification can be performed according to methods available in the art. (see, e.g., Godfrey et al. J. Molec. Diagnostics 2: 84-91 (2000); Specht et al., Am. J. Pathol. 158: 419-29 (2001)).

PCR primers and probes can be designed based upon exon or intron sequences present in the mRNA transcript of the gene of interest. Primer/probe design can be performed using publicly available software, such as the DNA BLAT software developed by Kent, W. J., Genome Res. 12(4):656-64 (2002), or by the BLAST software including its variations.

Where necessary or desired, repetitive sequences of the target sequence can be masked to mitigate non-specific signals. Exemplary tools to accomplish this include the Repeat Masker program available on-line through the Baylor College of Medicine, which screens DNA sequences against a library of repetitive elements and returns a query sequence in which the repetitive elements are masked. The masked intron sequences can then be used to design primer and probe sequences using any commercially or otherwise publicly available primer/probe design packages, such as Primer Express (Applied Biosystems); MGB assay-by-design (Applied Biosystems); Primer3 (Steve Rozen and Helen J. Skaletsky (2000) Primer3 on the WWW for general users and for biologist programmers. See S. Rrawetz, S. Misener, Bioinformatics Methods and Protocols: Methods in Molecular Biology, pp. 365-386 (Humana Press).

Other factors that can influence PCR primer design include primer length, melting temperature (Tm), and G/C content, specificity, complementary primer sequences, and 3′-end sequence. In general, optimal PCR primers are generally 17-30 bases in length, and contain about 20-80%, such as, for example, about 50-60% G+C bases, and exhibit Tm's between 50 and 80 OC, e.g. about 50 to 70° C.

For further guidelines for PCR primer and probe design see, e.g. Dieffenbach, C W. et al, “General Concepts for PCR Primer Design” in: PCR Primer, A Laboratory Manual, Cold Spring Harbor Laboratory Press. New York, 1995, pp. 133-155; Innis and Gelfand, “Optimization of PCRs” in: PCR Protocols, A Guide to Methods and Applications, CRC Press, London, 1994, pp. 5-11; and Plasterer, T. N. Primerselect: Primer and probe design. Methods MoI. Biol. 70:520-527 (1997), the entire disclosures of which are hereby expressly incorporated by reference.

In MassARRAY-based methods, such as the exemplary method developed by Sequenom, Inc. (San Diego, Calif.) following the isolation of RNA and reverse transcription, the obtained cDNA is spiked with a synthetic DNA molecule (competitor), which matches the targeted cDNA region in all positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivarion of the alkaline phosphatase, the PCR products from the competitor and cDNA are subjected to primer extension, which generates distinct mass signals for the competitor- and cDNA-derives PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see, e.g. Ding and Cantor, Proc. Natl. Acad. Sci. USA 100:3059-3064 (2003).

Further PCR-based techniques that can find use in the methods disclosed herein include, for example, BeadArray® technology (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression® (BADGE), using the commercially available LuminexlOO LabMAP® system and multiple color-coded microspheres (Luminex Corp., Austin, Tex.) in a rapid assay for gene expression (Yang et al., Genome Res. 11:1888-1898 (2001)); and high coverage expression profiling (HiCEP) analysis (Fukumura et al., Nucl. Acids. Res. 31(16) e94 (2003).

Expression levels of a gene or microArray of interest can also be assessed using the microarray technique. In this method, polynucleotide sequences of interest (including cDNAs and oligonucleotides) are arrayed on a substrate. The arrayed sequences are then contacted under conditions suitable for specific hybridization with detectably labeled cDNA generated from RNA of a test sample.

For example, PCR amplified inserts of cDNA clones of a gene to be assayed are applied to a substrate in a dense array. Usually at least 10,000 nucleotide sequences are applied to the substrate. For example, the microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After washing under stringent conditions to remove non-specifically bound probes, the chip is scanned by confocal laser microscopy or by another detection method, such as a CCD camera. Quantitation of hybridization of each arrayed element allows for assessment of corresponding RNA abundance.

With dual color fluorescence, separately labeled cDNA probes generated from two sources of RNA are hybridized pair wise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et at, Proc. Natl. Acad. ScL USA 93(2):106-149 (1996)). Microarray analysis can be performed by commercially available equipment, following manufacturer's protocols, such as by using the Affymetrix GenChip® technology, or Incyte's microarray technology.

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484-487 (1995); and Velculescu et al., Cell 88:243-51 (1997).

Nucleic acid sequencing technologies are suitable methods for analysis of gene expression. The principle underlying these methods is that the number of times a cDNA sequence is detected in a sample is directly related to the relative expression of the RNA corresponding to that sequence. These methods are sometimes referred to by the term Digital Gene Expression (DGE) to reflect the discrete numeric property of the resulting data. Early methods applying this principle were Serial Analysis of Gene Expression (SAGE) and Massively Parallel Signature Sequencing (MPSS). See, e.g., S. Brenner, et al., Nature Biotechnology 18(6):630-634 (2000). More recently, the advent of “next-generation” sequencing technologies has made DGE simpler, higher throughput, and more affordable. As a result, more laboratories are able to utilize DGE to screen the expression of more genes in more individual patient samples than previously possible. See, e.g., J. Marioni, Genome Research 18(9):1509-1517 (2008); R. Morin, Genome Research 18(4):610-621 (2008); A. Mortazavi, Nature Methods 5(7):621-628 (2008); N. Cloonan, Nature Methods 5(7):613-619 (2008).

In some embodiments, RNA extraction, sequencing, and analysis (transcriptomics) is performed as described in Example 1, although any suitable method known in the art may be utilized.

Methods of isolating RNA for expression analysis from blood, plasma and serum (see, e.g., K. Enders, et al., Clin Chem 48, 1647-53 (2002) (and references cited therein) and from urine (see, e.g., R. Boom, et al., J Clin Microbiol. 28, 495-503 (1990) and references cited therein) have been described.

Immunohistochemistry methods are suitable for detecting the expression levels of genes and applied to the method disclosed herein. Antibodies (e.g., monoclonal antibodies) that specifically bind a gene product of a gene of interest can be used in such methods. The antibodies can be detected by direct labeling of the antibodies themselves, for example, with radioactive labels, fluorescent labels, hapten' labels such as, biotin, or an enzyme such as horse radish peroxidase or alkaline phosphatase. Alternatively, unlabeled primary antibody can be used in conjunction with a labeled secondary antibody specific for the primary antibody. Immunohistochemistry protocols and kits are well known in the art and are commercially available.

The term “proteome” is defined as the totality of the proteins present in a sample (e.g. tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics.

One having ordinary skill in the art will recognize that there are many statistical methods that may be used to determine whether there is a significant relationship between a clinical outcome of interest (e.g., recurrence) and expression levels of a plurality of marker genes as described herein. Exemplary methods used for individual training and validation cohorts are described in Example 1. One of ordinary skill in the art will recognize that many methods now known or later developed will fall within the scope and spirit of the present disclosure. These methods may incorporate, for example, correlation coefficients, co-expression network analysis, etc., and may be based on expression data from RT-PCR, microarrays, sequencing, and other similar technologies. For example, gene expression analysis can be identified using pair-wise analysis of correlation based on Pearson or Spearman correlation coefficients. (See, e.g., Pearson K. and Lee A., Biometrika 2, 357 (1902); C. Spearman, Amer. J. Psychol 15:72-101 (1904); J. Myers, A. Well, Research Design and Statistical Analysis, p. 508 (2nd Ed., 2003).)

The expression data used in the methods disclosed herein can be normalized. In some embodiments, methods of determining the expression level of a plurality (e.g., three, four, five, six, seven, eight, nine, ten, eleven, twelve, thirteen, fourteen, 14-20, 20-40, 40-60, 60-80, 80-100 or more) of markers from the markers shown in FIG. 6 comprises determining the expression level of the plurality of markers in non-phagocytic and/or CD2+ cells of the sample as well as in phagocytic and/or CD14+ cells. In some embodiments, the methods further comprise normalizing the expression level of each of the markers via determining the log ratio of CD14+ over CD2+ expression levels. Those skilled in the art will recognize that normalization may be achieved in numerous ways, and the techniques described above are intended only to be exemplary, not exhaustive.

In some aspects, the disclosure utilizes compositions and methods described herein and/or one or more immunocyte transcriptomic signature models identified herein to stratify cancer patients. For example, in one embodiment, the disclosure provides assays and/or one or more immunocyte transcriptomic signature models identified herein to stratify patients with indolent prostate disease from those with aggressive prostate cancer (e.g., that require life-saving treatments). Accordingly, in some embodiments, compositions and methods described herein find use in clinical assessment and management of subjects (e.g., patients at risk for cancer (e.g., prostate cancer)). For example, in some embodiments, assays and/or one or more signatures identified herein classify a patient as definitive for treatment (e.g., with one or more anti-cancer therapies) or as needing only surveillance (e.g., active surveillance and/or no treatment).

In some embodiments, compositions, and methods of the disclosure (e.g., assays and/or one or more immunocyte transcriptomic signature models identified herein) provide a clinician the ability to stratify a patient into either a treatment group (e.g., requiring cancer treatment and/or therapies) or a surveillance group (e.g., not requiring immediate treatment) without need for a physically invasive biopsy. That is, in some embodiments, compositions and methods of the disclosure are used to avoid unnecessary patient biopsies (e.g., prostate cancer biopsy), repeat biopsies, and/or the pain and suffering and risk factors/side effects consequent to biopsies (e.g., in men under heretofore conventional active surveillance for prostate cancer). In some embodiments, compositions and methods of the disclosure benefit men diagnosed with prostate cancer in that the compositions and methods (assays and/or one or more signatures identified herein) identify patients needing further workup and/or treatment.

In one embodiment, the present disclosure provides biological markers and methods of using them to detect a cancer (e.g., prostate cancer). The present disclosure is based on the discovery that one or more markers described in FIG. 6 are useful in diagnosing prostate cancer, either alone, or when assessed in the context of one or more clinical covariates (e.g., age, PSAD, PSA, etc.).

The markers of this disclosure can be used in methods for diagnosing or aiding in the diagnosis of prostate cancer by comparing levels (e.g., gene expression levels, or protein expression levels, or protein activities) of one or more prostate cancer markers (e.g., nucleic acids or proteins) between phagocytes (e.g., macrophages, monocytes, or neutrophils) and non-phagocytic cells (e.g., T cells) or a cell free component taken from the same individual. This disclosure also provides methods for assessing the risk of developing prostate cancer, prognosing the cancer, monitoring the cancer progression or regression, assessing the efficacy of a treatment, or identifying a compound capable of ameliorating or treating the cancer.

In some embodiments, the disclosure provides a method of measuring a panel of biomarkers in a subject comprising obtaining a biological sample from the subject; determining a measurement for the panel of biomarkers in the biological sample, wherein the panel of biomarkers comprise one or more (e.g., two, three, four, five, six, seven, eight, nine, ten, eleven, twelve, thirteen, fourteen, fifteen or more) biomarkers of FIG. 6 and wherein the measurement comprises measuring a level of each of the biomarkers in the panel. In some embodiments, measuring the panel of biomarkers in the subject identifies, assesses, and/or predicts the aggressiveness or indolence of cancer (e.g., prostate cancer) in a subject (e.g., a subject suspected of having cancer, a subject diagnosed with a cancer, or a subject at risk for cancer). In some embodiments, the biological sample comprises CD2+ cells and/or CD14+ cells. In one embodiment, determining a measurement for the panel of biomarkers in the biological sample comprises measuring a level of each of the biomarkers in the panel in CD2+ cells and/or CD14+ cells.

The disclosure is not limited by how gene expression levels are measured. Indeed, any means of measuring gene expression levels may be used including, but not limited to, polymerase chain reaction (PCR) analysis, sequencing analysis, electrophoretic analysis, restriction fragment length polymorphism (RFLP) analysis, Northern blot analysis, quantitative PCR, reverse-transcriptase-PCR analysis (RT-PCR), allele-specific oligonucleotide hybridization analysis, comparative genomic hybridization, heteroduplex mobility assay (HMA), single strand conformational polymorphism (SSCP), denaturing gradient gel electrophoresis (DGGE), RNAase mismatch analysis, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), surface plasmon resonance, Southern blot analysis, in situ hybridization, fluorescence in situ hybridization (FISH), chromogenic in situ hybridization (CISH), immunohistochemistry (IHC), microarray, comparative genomic hybridization, karyotyping, multiplex ligation-dependent probe amplification (MLPA), Quantitative Multiplex PCR of Short Fluorescent Fragments (QMPSF), microscopy, methylation specific PCR (MSP) assay, HpaII tiny fragment Enrichment by Ligation-mediated PCR (HELP) assay, radioactive acetate labeling assays, colorimetric DNA acetylation assay, chromatin immunoprecipitation combined with microarray (ChIP-on-chip) assay, restriction landmark genomic scanning, Methylated DNA immunoprecipitation (MeDIP), molecular break light assay for DNA adenine methyltransferase activity, chromatographic separation, methylation-sensitive restriction enzyme analysis, bisulfite-driven conversion of non-methylated cytosine to uracil, methyl-binding PCR analysis, or a combination thereof. In some embodiments, gene expression levels are measured by a sequencing technique such as, but not limited to, direct sequencing, RNA sequencing, whole transcriptome shotgun sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, mass spectrometry, and a combination thereof. In some embodiments, measuring a level of each of the biomarkers in the panel comprises measuring protein expression levels.

The disclosure is not limited to any particular method of measuring protein expression levels. Exemplary methods of measuring protein expression levels include, but are not limited to, an immunohistochemistry assay, an enzyme-linked immunosorbent assay (ELISA), in situ hybridization, chromatography, liquid chromatography, size exclusion chromatography, high performance liquid chromatography (HPLC), gas chromatography, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), radioimmunoassays, microscopy, microfluidic chip-based assays, surface plasmon resonance, sequencing, Western blotting assay, or a combination thereof. In some embodiments, measuring a level of each of the biomarkers in the panel comprises measuring by a qualitative assay, a quantitative assay, or a combination thereof. Exemplary quantitative assays include, but are not limited to, sequencing, direct sequencing, RNA sequencing, whole transcriptome shotgun sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), polymerase chain reaction (PCR) analysis, quantitative PCR, real-time PCR, fluorescence assay, colorimetric assay, chemiluminescent assay, or a combination thereof.

In certain embodiments, methods of this disclosure also comprise at least one of the following steps before determination of various levels: i) lysing the phagocytic or non-phagocytic cells; and ii) extracting cellular contents from the lysed cells. Any known cell lysis and extraction methods can be used herein. In certain embodiments, at least one or more prostate cancer markers are present in the phagocytes. In certain embodiments, there is no marker present in the cellular contents of the non-phagocytic cells.

In certain embodiments, the phagocytic cells and/or non-phagocytic cells are isolated from a bodily fluid sample, tissues, or population of cells. Exemplary bodily fluid samples can be whole blood, urine, stool, saliva, lymph fluid, cerebrospinal fluid, synovial fluid, cystic fluid, ascites, pleural effusion, fluid obtained from a pregnant woman in the first trimester, fluid obtained from a pregnant woman in the second trimester, fluid obtained from a pregnant woman in the third trimester, maternal blood, amniotic fluid, chorionic villus sample, fluid from a preimplantation embryo, maternal urine, maternal saliva, placental sample, fetal blood, lavage and cervical vaginal fluid, interstitial fluid, buccal swab sample, sputum, bronchial lavage, Pap smear sample, or ocular fluid. In some embodiments, the phagocytic cells or non-phagocytic cells are isolated from white blood cells.

In the methods of this disclosure, cell separation/isolation/purification methods are used to isolate populations of cells from bodily fluid sample, cells, or tissues of a subject. A skilled worker can use any known cell separation/isolation/purification techniques to isolate phagocytic cells and non-phagocytic cells from a bodily fluid. Exemplary techniques include, but are not limited to, using antibodies, flow cytometry, fluorescence activated cell sorting, filtration, gradient-based centrifugation, elution, microfluidics, immunomagnetic separation technique, multiple size immuno-beads filtration techniques, fluorescent-magnetic separation technique, nanostructure, quantum dots, high throughput microscope-based platform, or a combination thereof.

In certain embodiments, the phagocytic cells and/or non-phagocytic cells are isolated by using a product secreted by the cells. In certain embodiments, the phagocytic cells and/or non-phagocytic cells are isolated by using a cell surface target (e.g., receptor protein) on the surface of the cells. In some embodiments, the cell surface target is a protein that has been engulfed by phagocytic cells. In some embodiments, the cell surface target is expressed by cells on their plasma membranes. In some embodiments, the cell surface target is an exogenous protein that is translocated on the plasma membranes, but not expressed by the cells (e.g., the phagocytic cells). In some embodiments, the cell surface target is a marker of prostate cancer.

In certain aspects of the methods described herein, analytes include nucleic acids, proteins, or any combinations thereof. In certain aspects of the methods described herein, markers include nucleic acids, proteins, or any combinations thereof. As used herein, the term “nucleic acid” is intended to include DNA molecules (e.g., cDNA or genomic DNA), RNA molecules (e.g., mRNA), DNA-RNA hybrids, and analogs of the DNA or RNA generated using nucleotide analogs. The nucleic acid molecule can be a nucleotide, oligonucleotide, double-stranded DNA, single-stranded DNA, multi-stranded DNA, complementary DNA, genomic DNA, non-coding DNA, messenger RNA (mRNAs), microRNA (miRNAs), small nucleolar RNA (snoRNAs), ribosomal RNA (rRNA), transfer RNA (tRNA), small interfering RNA (siRNA), heterogeneous nuclear RNAs (hnRNA), or small hairpin RNA (shRNA). In some embodiments, the nucleic acid is a transrenal nucleic acid. A transrenal nucleic acid is an extracellular nucleic acid that is excreted in the urine. See, e.g., U.S. Patent Publication No. 20100068711 and U.S. Patent Publication No. 20120021404.

As used herein, the term “amino acid” includes organic compounds containing both a basic amino group and an acidic carboxyl group. Included within this term are natural amino acids (e.g., L-amino acids), modified and unusual amino acids (e.g., D-amino acids and .beta.-amino acids), as well as amino acids which are known to occur biologically in free or combined form but usually do not occur in proteins. Natural protein occurring amino acids include alanine, arginine, asparagine, aspartic acid, cysteine, glutamic acid, glutamine, glycine, histidine, isoleucine, leucine, lysine, methionine, phenylalanine, serine, threonine, tyrosine, tryptophan, proline, and valine. Natural non-protein amino acids include arginosuccinic acid, citrulline, cysteine sulfuric acid, 3,4-dihydroxyphenylalanine, homocysteine, homoserine, ornithine, 3-monoiodotyrosine, 3,5-diiodotryosine, 3,5,5-triiodothyronine, and 3,3′,5,5′-tetraiodothyronine. Modified or unusual amino acids include D-amino acids, hydroxylysine, 4-hydroxyproline, N-Cbz-protected amino acids, 2,4-diaminobutyric acid, homoarginine, norleucine, N-methylaminobutyric acid, naphthylalanine, phenylglycine, .alpha.-phenylproline, tert-leucine, 4-aminocyclohexylalanine, N-methyl-norleucine, 3,4-dehydroproline, N,N-dimethylaminoglycine, N-methylaminoglycine, 4-aminopiperidine-4-carboxylic acid, 6-aminocaproic acid, trans-4-(aminomethyl)-cyclohexanecarboxylic acid, 2-, 3-, and 4-(aminomethyl)-benzoic acid, 1-aminocyclopentanecarboxylic acid, 1-aminocyclopropanecarboxylic acid, and 2-benzyl-5-aminopentanoic acid.

As used herein, the term “peptide” includes compounds that consist of two or more amino acids that are linked by means of a peptide bond. Peptides may have a molecular weight of less than 10,000 Daltons, less than 5,000 Daltons, or less than 2,500 Daltons. The term “peptide” also includes compounds containing both peptide and non-peptide components, such as pseudopeptide or peptidomimetic residues or other non-amino acid components. Such compounds containing both peptide and non-peptide components may also be referred to as a “peptide analog.”

As used herein, the term “protein” includes compounds that consist of amino acids arranged in a linear chain and joined together by peptide bonds between the carboxyl and amino groups of adjacent amino acid residues. Proteins used in methods of the disclosure include, but are not limited to, amino acids, peptides, antibodies, antibody fragments, cytokines, lipoproteins, or glycoproteins.

As used herein, the term “antibody” includes polyclonal antibodies, monoclonal antibodies (including full length antibodies which have an immunoglobulin Fc region), antibody compositions with polyepitopic specificity, multispecific antibodies (e.g., bispecific antibodies, diabodies, and single-chain molecules, and antibody fragments (e.g., Fab or F(ab′).sub.2, and Fv). For the structure and properties of the different classes of antibodies, see e.g., Basic and Clinical Immunology, 8th Edition, Daniel P. Sties, Abba I. Ten and Tristram G. Parsolw (eds), Appleton & Lange, Norwalk, Conn., 1994, page 71 and Chapter 6.

As used herein, the term “sequencing” is used in a broad sense and refers to any technique known in the art that allows the order of at least some consecutive nucleotides in at least part of a nucleic acid to be identified, including without limitation at least part of an extension product or a vector insert. Exemplary sequencing techniques include targeted sequencing, single molecule real-time sequencing, whole transcriptome shotgun sequencing (“RNA-seq”), electron microscopy-based sequencing, transistor-mediated sequencing, direct sequencing, random shotgun sequencing, Sanger dideoxy termination sequencing, exon sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, mass spectrometry, and a combination thereof. In some embodiments, sequencing comprises an detecting the sequencing product using an instrument, for example but not limited to an ABI PRISM™ 377 DNA Sequencer, an ABI PRISM™ 310, 3100, 3100-Avant, 3730, or 3730xI Genetic Analyzer, an ABI PRISM™ 3700 DNA Analyzer, or an Applied Biosystems SOLiD™ System (all from Applied Biosystems), a Genome Sequencer 20 System (Roche Applied Science), or a mass spectrometer. In certain embodiments, sequencing comprises emulsion PCR. In certain embodiments, sequencing comprises a high throughput sequencing technique, for example but not limited to, massively parallel signature sequencing (MPSS).

In further embodiments of the disclosure, a protein level can be a protein expression level, a protein activation level, or a combination thereof. In some embodiments, a protein activation level can comprise determining a phosphorylation state, an ubiquitination state, a myristylation state, or a conformational state of the protein.

A protein level can be detected by any methods known in the art for detecting protein expression levels, protein phosphorylation state, protein ubiquitination state, protein myristylation state, or protein conformational state. In some embodiments, a protein level can be determined by an immunohistochemistry assay, an enzyme-linked immunosorbent assay (ELISA), in situ hybridization, chromatography, liquid chromatography, size exclusion chromatography, high performance liquid chromatography (HPLC), gas chromatography, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), radioimmunoassays, microscopy, microfluidic chip-based assays, surface plasmon resonance, sequencing, Western blotting assay, or a combination thereof.

As used herein, the “difference” between different levels detected by the methods of this disclosure can refer to different gene copy numbers, different DNA, RNA, or protein expression levels, different DNA methylation states, different DNA acetylation states, and different protein modification states. The difference can be a difference greater than 1 fold (e.g., 1.0 to 100.0 fold, or greater). In some embodiments, the difference is a 1.05-fold, 1.1-fold, 1.2-fold, 1.3-fold, 1.4-fold, 1.5-fold, 2-fold, 2.5-fold, 3-fold, 4-fold, 5-fold, 6-fold, 7-fold, 8-fold, 9-fold, 10-fold pr more difference. In some embodiments, the difference is any fold difference between 1-10, 2-10, 5-10, 10-20, or 10-100 fold.

The methods of the disclosure can also be used to detect genetic alterations in a marker gene, thereby determining if a subject with the altered gene is at risk for developing prostate cancer characterized by misregulation in a marker protein activity or nucleic acid expression. In certain embodiments, the methods include detecting, in phagocytes, the presence or absence of a genetic alteration characterized by an alteration affecting the integrity of a gene encoding a marker peptide and/or a marker gene. For example, such genetic alterations can be detected by ascertaining the existence of at least one of: 1) a deletion of one or more nucleotides from one or more marker genes; 2) an addition of one or more nucleotides to one or more marker genes; 3) a substitution of one or more nucleotides of one or more marker genes, 4) a chromosomal rearrangement of one or more marker genes; 5) an alteration in the level of a messenger RNA transcript of one or more marker genes; 6) aberrant modification of one or more marker genes, such as of the methylation pattern of the genomic DNA; 7) the presence of a non-wild type splicing pattern of a messenger RNA transcript of one or more marker genes; 8) a non-wild type level of a one or more marker proteins; 9) allelic loss of one or more marker genes; and 10) inappropriate post-translational modification of one or more marker proteins. As described herein, there are a large number of assays known in the art which can be used for detecting alterations in one or more marker genes.

In certain embodiments, detection of the alteration involves the use of a probe/primer in a polymerase chain reaction (PCR) (see, e.g., U.S. Pat. Nos. 4,683,195, 4,683,202 and 5,854,033), such as real-time PCR, COLD-PCR (Li et al. (2008) Nat. Med. 14:579), anchor PCR, recursive PCR or RACE PCR, or, alternatively, in a ligation chain reaction (LCR) (see, e.g., Landegran et al. (1988) Science 241:1077; Prodromou and Pearl (1992) Protein Eng. 5:827; and Nakazawa et al. (1994) Proc. Natl. Acad. Sci. USA 91:360), the latter of which can be particularly useful for detecting point mutations in a marker gene (see Abravaya et al. (1995) Nucleic Acids Res. 23:675). This method can include the steps of collecting a sample of cell free bodily fluid from a subject, isolating nucleic acid (e.g., genomic, mRNA or both) from the sample, contacting the nucleic acid sample with one or more primers which specifically hybridize to a marker gene under conditions such that hybridization and amplification of the marker gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self-sustained sequence replication (Guatelli et al., (1990) Proc. Natl. Acad. Sci. USA 87:1874), transcriptional amplification system (Kwoh et al., (1989) Proc. Natl. Acad. Sci. USA 86:1173), Q Beta Replicase (Lizardi et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in one or more marker genes from a sample can be identified by alterations in restriction enzyme cleavage patterns. For example, sample and control DNA is isolated, optionally amplified, digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in one or more of the markers described herein can be identified by hybridizing a sample and control nucleic acids, e.g., DNA or RNA, to high density arrays containing hundreds or thousands of oligonucleotides probes (Cronin et al. (1996) Human Mutation 7: 244; Kozal et al. (1996) Nature Medicine 2:753). For example, genetic mutations in a marker nucleic acid can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence a marker gene and detect mutations by comparing the sequence of the sample marker gene with the corresponding wild-type (control) sequence. Examples of sequencing reactions include those described herein as well as those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147).

Other methods for detecting mutations in a marker gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type marker sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397; Saleeba et al. (1992) Methods Enzymol. 217:286. In one embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in marker cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657). According to an exemplary embodiment, a probe based on a marker sequence, e.g., a wild-type marker sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in marker genes. For example, single strand conformation polymorphism (SSCP) may be used to detect differences in electrophoretic mobility between mutant and wild type nucleic acids (Orita et al. (1989) Proc. Natl. Acad. Sci. USA 86:2766, see also Cotton (1993) Mutat. Res. 285:125; and Hayashi (1992) Genet. Anal. Tech. Appl. 9:73). Single-stranded DNA fragments of sample and control marker nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification or selective primer extension. For example, oligonucleotide primers may be prepared in which the known mutation is placed centrally and then hybridized to target DNA under conditions which permit hybridization only if a perfect match is found (Saiki et al. (1986) Nature 324:163; Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant disclosure. Oligonucleotides used as primers for specific amplification may carry the mutation of interest in the center of the molecule (so that amplification depends on differential hybridization) (Gibbs et al. (1989) Nucl. Acids Res. 17:2437) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The genes/biomarkers useful in the compositions and methods of the disclosure can include any mutation in any one of the genes/biomarkers. Mutation sites and sequences can be identified, for example, by databases or repositories of such information, e.g., The Human Gene Mutation Database (United Kingdom), the Single Nucleotide Polymorphism Database (dbSNP, NCBI, USA), and the Online Mendelian Inheritance in Man (OMIM, NCBI, USA).

The one or more genes/biomarkers identified by this disclosure (e.g., markers in FIG. 6) may be used in the treatment of prostate cancer. For example, a marker (e.g., a protein or gene) identified by the present disclosure may be used as a molecular target for a therapeutic agent. A marker identified by the disclosure also may be used in any of the other methods of the disclosure, e.g., for monitoring the progression or regression of a disease or condition. In certain embodiments, the one or more markers identified by the methods of this disclosure may have therapeutic potential. For example, if a marker is identified as being up-regulated (or down-regulated), or activated (or inhibited) in phagocytic cells from a subject having prostate cancer, a compound or an agent that is capable of down-regulating (or up-regulating) or inhibiting (or activating) said marker may be useful in treating prostate cancer. Similarly, a gene protein expression level, a protein expression level, or a combination thereof may be useful in this aspect of the disclosure.

Materials for use in the methods of the present disclosure are suited for preparation of kits produced in accordance with well-known procedures. The present disclosure thus provides kits comprising agents, which may include gene-specific or gene-selective probes and/or primers, for quantifying the expression of the disclosed genes for predicting prognostic outcome or response to treatment. Such kits may optionally contain reagents for the extraction of RNA from tumor samples. In addition, the kits may optionally comprise the reagent(s) with an identifying description or label or instructions relating to their use in the methods of the present disclosure. The kits may comprise containers (including microliter plates suitable for use in an automated implementation of the method), each with one or more of the various materials or reagents (typically in concentrated form) utilized in the methods, including, for example, chromatographic columns, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more probes and primers of the present disclosure (e.g., appropriate length poly(T) or random primers linked to a promoter reactive with the RNA polymerase). Mathematical algorithms used to estimate or quantify prognostic or predictive information are also properly potential components of kits.

The compositions and methods of this disclosure, when practiced for commercial diagnostic purposes, generally produce a report or summary of information obtained from the herein-described compositions and methods. For example, a report may include information concerning a patient's ASRS, expression levels of one or more genes, classification of the tumor or the patient's risk of recurrence, the patient's likely prognosis, clinical and pathologic factors, and/or other information. The methods and reports of this disclosure can further include storing the report in a database. The method can create a record in a database for the subject and populate the record with data. The report may be a paper report, an auditory report, or an electronic record. The report may be displayed and/or stored on a computing device (e.g., handheld device, desktop computer, smart device, website, etc.). It is contemplated that the report is provided to a physician and/or the patient. The receiving of the report can further include establishing a network connection to a server computer that includes the data and report and requesting the data and report from the server computer.

The values and/or scores from the methods described here, such as ASRS, can be calculated and stored manually. Alternatively, the above-described steps can be completely or partially performed by a computer program product. The present disclosure thus provides a computer program product including a computer readable storage medium having a computer program stored on it. The program can, when read by a computer, execute relevant calculations based on values obtained from analysis of one or more biological samples from an individual (e.g., gene expression levels, normalization, and/or conversion of values from assays to a score (e.g., ASRS) and/or text or graphical depiction of risk score, AS status and/or recommendation, tumor stage and related information). The computer program product has stored therein a computer program for performing the calculation.

The present disclosure provides systems for executing the program described above, which system generally includes: a) a central computing environment; b) an input device, operatively connected to the computing environment, to receive patient data, wherein the patient data can include, for example, expression level or other value obtained from an assay using a biological sample from the patient, as described in detail herein; c) an output device, connected to the computing environment, to provide information to a user (e.g., medical personnel); and d) an algorithm executed by the central computing environment (e.g., a processor), where the algorithm is executed based on the data received by the input device, and wherein the algorithm calculates an ASRS or other functions described herein. The methods provided by the present disclosure may also be automated in whole or in part.

Unless otherwise defined herein, scientific and technical terms used in this application shall have the meanings that are commonly understood by those of ordinary skill in the art. Generally, nomenclature used in connection with, and techniques of, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The following examples are set forth as being representative of the present disclosure. These examples are not to be construed as limiting the scope of the disclosure as these and other equivalent embodiments will be apparent to one of ordinary skill in the art in view of the present disclosure and accompanying claims.

The disclosure may be embodied in other specific forms without departing from the spirit or essential characteristics thereof. The foregoing embodiments are therefore to be considered in all respects illustrative rather than limiting the disclosure described herein. Scope of the disclosure is thus indicated by the appended claims rather than by the foregoing description, and all changes that come within the meaning and range of equivalency of the claims are intended to be embraced therein. All publications, patents, and patent applications cited herein are hereby incorporated by reference in their entirety for all purposes

### EXAMPLES

The following examples illustrate but do not limit the compounds, compositions, and methods of the present disclosure. Other suitable modifications and adaptations of the variety of conditions and parameters normally encountered in clinical therapy and which are obvious to those skilled in the art are within the spirit and scope of the disclosure.

### Example 1. Training and Validation Cohorts Utilizing Subtraction-Normalized Immunocyte Signature Profiling

Studies were performed to identify genes and expression levels thereof useful in algorithm-based assays that involve subtraction-normalized immunocyte signature profiling from a sample obtained from a prostate cancer patient. This example provides and characterizes blood-based immunocyte transcriptomic signatures to identify men harboring occult aggressive prostate cancer (PCa) and validate it on a biopsy-positive population to identify men who should not be on active surveillance (AS) and confirm those men with indolent disease who can safely remain on AS. This model uses subtraction-normalized immunocyte transcriptomic profiles to risk stratify men with PCa that are candidates for AS. This model was validated in an independent cohort disclosed herein.

### Materials and Methods

Patient populations. The models presented in this disclosure were developed and validated on two large independent cohorts of men who were visiting their urologists because of suspected PCa or were known to have untreated PCa and signed an informed consent. Patients in the training cohort were enrolled from Comprehensive Urology (Metropolitan Detroit, Michigan), Michigan Institute of Urology (Metropolitan Detroit, Michigan), and Urology Austin (Austin, Texas), with specimens collected between 16 Oct. 2013 through 30 Oct. 2014, and were used to train a model for AS purposes. In addition to the three clinical sites of the training cohort, patients from Associated Medical Professionals (Syracuse, New York), The Urology Center of Colorado (Denver, Colorado), and Urology Associates (Nashville, Tennessee) were also included in the independent validation cohort, with specimens collected between 10 Aug. 2017 through 14 Aug. 2020. The biopsy-positive subset of the independent validation cohort served as a surrogate AS population.

Men were eligible for enrollment in the study if they (i) were determined by their physician to have a risk profile that warranted a prostate biopsy, or (ii) had a biopsy >90 days prior to but <1 year of study entry and had not undergone definitive therapy, or (iii) were on AS after the diagnosis of PCa such that a biopsy would be performed within the next year. Men were excluded from enrollment in this study if they (i) were less than 40 years of age, (ii) had any known concurrent cancer except non-melanoma skin cancer or any history of cancer in the last 5 years, or (iii) had any form of androgen deprivation therapy (ADT) with the exception of 5-alpha reductase inhibitors.

Clinical, laboratory, and pathology data of each patient was abstracted from the electronic medical record and entered into an electronic data capture (EDC) system by the research teams at the various institutions under IRB approved protocols. Standard 12-core systematic biopsies were performed with allowance for additional cores at the urologist's discretion, while some patients proceeded to have a radical prostatectomy. The current International Society of Urological Pathology (ISUP) modified Gleason grading system was used. Specimen data from all follow-up procedures was used to classify patients, with a median follow-up of 25 months for the validation cohort. Cases of aggressive disease were defined as NCCN unfavorable intermediate classification, or worse, for tissue and T-stage criteria based on needle biopsies. An extrapolation was applied for radical prostatectomies, multiplying the tumor length in mm by a factor 3 to obtain a surrogate measure for number of cores positive (with a maximum of 12).

Blood Collection and Processing. Blood was collected from all eligible patients participating in the study consequent to prostate biopsy. Sample of the training cohort were collected as described¹⁹. Blood samples of the independent validation set were collected in CPT BD Vacutainer™ tubes (Cat. No. 362761, BD Biosciences, San Jose, California). Samples were centrifuged on site according to manufacturer's instructions to isolate peripheral blood mononuclear cells (PBMCs). Samples were transported and stored at 4° C. for processing within 72 hours of the blood draw. PBMCs from each individual were pooled through a 70 μm filter at 4° C. into a single tube. Samples were split into approximately ⅓ and ⅔ aliquots for CD2 and CD14 cell type isolations. Aliqutos were centrifuged at 300×g for 10 minutes at room temperature to produce cell pellets. The supernatant was discarded, and cells were suspended in 225 μl for CD2, and 400 μl for CD14, 4° C. autoMACS running buffer (Cat. No. 130-091-221, Miltenyi Biotech, Bergisch Gladbach, Germany). Specially formulated positive selection MACS Microbeads using anti-CD2 antibodies and anti-CD14 antibodies (Cat. No. 130-091-114 and 130-118-906, respectively, Miltenyi Biotech) were added to the aliquots of PBMCs at a volume of 25 μl CD2 beads and 100 μl CD14 beads. Beads and cells were incubated together for 15 minutes at 4° C. After the 15-minute incubation 250 μl of 4° C. auto MACS running buffer was added to the CD2 sample to bring both samples to a total volume of 500 μl. Samples were processed using a positive selection template on the Multi MACS™ Cell24 Separator Plus (Miltenyi Biotech) to isolate CD2 and CD14 cells. Total PSA of patient serum samples was measured using the Cobas e 411 system from Roche. Serum was collected using Greiner Bio-One VACUETTE™ Z Serum Sep Clot Activator Tubes (Greiner Bio-One, Cat #456073P), which were centrifuged on site to separate the serum from the rest of the blood components. Serum was transferred to the laboratory at 4° C. and stored at −80° C. until time of analysis.

RNA Extraction and Sequencing. RNA extraction was performed on the KingFisher Flex instrument (Cat. No. 50-152-7925, Fisher Scientific) using the Maxwell® HT simplyRNA Kit (Cat. No. AX7890, Promega). The simplyRNA kit and the KingFisher program were modified to optimize RNA extraction from eluted cells after cell separation. RNA samples were quantified using the Quan-iT RiboGreen RNA Assay (Invitrogen) and the RNA integrity was assessed by electrophoresis using the Tape Station system (Agilent). Samples were required to have a RIN number higher than 7 to proceed with the next steps. A minimum of 300 ng of total RNA was used as template for the library construction. Libraries were constructed using the Universal Plus mRNA-Seq library preparation kit with NuQuant (Tecan). The molar concentration of each individual library was determined by fluorescence using a Qbit fluorometer (Invitrogen) and the corresponding NuQuant standards. Some libraries across the study were also quantified with more traditional methods using fragment size analysis (Bioanalyzer, Invitrogen) and qPCR to correlate the results obtained using NuQuant. Libraries were combined in equimolar proportions generating 10 mM library pools. All pools were pre-run using iSeq 100 (Illumina) instrument to assure all libraries were present in comparable proportions and contributing equivalently to the final sequencing output. Pools were sequenced using a 100 bp paired-end mode in a NovaSeq 6000 sequencer (Illumina).

Transcriptomics. Raw sequencing reads in fastq format were quality-filtered using Seqpurge version 2019_11, also removing adapter sequences²⁰. Trimmed reads were mapped to the human genome reference (GRCh38, Ensembl version 100) with STAR version 2.7.2b using a 2-pass alignment with default parameters generating BAM files²¹. Quantification of the reads at gene level was performed from the BAM files using featureCounts version 2.0.1, considering only reads mapped to exons²². Quality reports after each step (raw, trimmed, and mapped reads) were generated using FastQC version 0.11.9 and aggregated in a final report using MultiQC version 1.9^{23,24}. After quantification, 24342 transcripts without gene symbol and 2870 mitochondrial and ribosomal transcripts were excluded using regular expressions, resulting in 33467 transcripts, from which 31918 common gene symbols between both cohorts with nonzero counts in at least one of the cell types (CD2 or CD14) were kept in the training and validation sets, respectively. Read depths were comparable across cell types, but different between cohorts, namely, 30.2±6.9 and 35.3±7.9 million median reads±standard deviation in the training cohort for CD2 and CD14, respectively, and 60.4±22.0 and 67.4±16.3 million median reads in the validation cohort, respectively. Four samples with cell-type-specific expression profiles that were inconsistent with population estimates via principal component analysis were excluded from downstream analyses. Normalization of raw counts was carried out via trimmed mean of M-values (TMM)²⁵. The log ratio of CD14 over CD2 reads was used to obtain intra-individual normalized counts¹⁹. Population differences in gene expression profiles between cohorts (i.e., batch effects) were addressed by simply transferring the gene-wise population means from the training into the validation cohort.

Data Analysis. Performance was evaluated in terms of the receiver operating characteristic (ROC) and its area (AUC)²⁶. Statistical significance of the difference between ROCs and AUC values for different models as well as 95% confidence intervals (CIs) were quantified by DeLong's method²⁷. Statistical comparisons for demographics and clinical covariates were carried out using chi-squared test for discrete covariates and Wilcoxon ranked sum test for continuous covariates. All results were produced using R version 4.2.2. All regularized models were fit using the glmnet 4.1-6 package and pathway analysis was performed with the enrichR package focusing on KEGG and WikiPathways^{28,29}. Missing values for total PSA and prostate volume were imputed to the median, and PSA density values were calculated after imputation. Transcript counts are aggregated at the gene symbol level and transcripts with zero reads for all samples were excluded.

Modeling. A model was created for the National Comprehensive Cancer Network (NCCN) endpoint consisting of a regularized logistic regression model³⁰, whose inputs are subsets of log-transformed count ratios of CD14 and CD2 values, with and without the clinicodemographic parameters age and PSA density. The model was less prone to overfitting using ranked subsets of the complete set of 31918 transcripts. The regularization parameter of the model controlling sparsity and the size of the ranked subset of transcripts were selected via 10-fold cross-validation on the complete training cohort. To select the ranked subset, all genes/biomarkers in the training cohort were first ranked by variance of the CD14 and CD2 ration, and then subsets of size 100 to 10000, in steps of 200 (e.g., the 100 genes/biomarkers with largest variance, then the top 200, . . . ). The model trained with the ranked subset and regularization parameter (for all possible regularization parameters via the least angle regression method) that yielded the best cross-validated performance in terms of AUC was selected for validation. Uusing small and large ranked subsets resulted in under- and over-fitting, respectively, thus motivating the need to select the best ranked subset size by cross-validation. Moreover, models both on the entire training cohort as well as only using the positive biopsies were built and it was noted that building the model on the entire training cohort yielded better cross-validated results in comparison to only using the biopsy-positive subset, which reduced the cohort in about half (See FIG. 4). Sample weighting during model fitting was effective at alleviating the potential bias induced by having a disproportionate amount of biopsy negative and low GG individuals (See distribution in FIG. 4). For the latter, a propensity weighting model was built using all transcripts as imputs, consisting of a regularized regression model using GG as an endpoint on the training cohort data. Propensity weights obtained via 10-fold cross-validation on the entire training set were calculated as the sigmoid-transformed outputs of the regression models. The sigmoid function was used to map regression values to the unit (0,1) interval. The model for NCCN fit with sample weights was applied once to the biopsy-positive subset of the independent validation cohort. Propensity weights were only used when fitting the model, thus were not required for model evaluation on the validation data. In some embodiments, a model combining the immunocyte transcriptomic signature with age and PSA density, was compared with a model consisting of these two clinical risk factors alone in the biopsy-positive subset of the validation cohort to evaluate potential synergy between risk factors.

Results. Independent Training and Validation Cohorts. For the training cohort, patient samples were collected from three urology centers, and three additional centers were used to collect patients for the independent validation cohort. The clinical and demographic characteristics of these two cohorts are shown in FIG. 4. Due to the changes in the clinical use of PSA as a screening biomarker over time, some minor changes in clinicodemographics were observed. Patients in the validation cohort were on average 11 months older at the time of diagnosis compared to those in the earlier training cohort. Similarly, overall PSA levels were 0.6 ng/mL higher in line with a less rigorous use of PSA as a screening tool. This is also noticeable in the lower number of men without PCa in the validation cohort, albeit that these additional cancers found are mainly of GG group 1 or 2.

Recent reviews advocate including select men with intermediate risk disease as eligible for AS³¹. Men were categorized according to the NCCN guidelines, with unfavorable intermediate or worse disease found on any biopsy after blood draw was considered aggressive disease that would result in ineligibility for AS. Because NCCN is also used to classify patients, metrics like PSA were not included since this would result in overfitting towards PSA. This modified NCCN definition of aggressiveness included clinical T-stage, GG, and percentage of positive cores. Aggressive disease was defined as disease stage ≥T2b and ≥50% of cores positive, or GG2 and ≥T2b, or GG2 and ≥50% of cores positive, or ≥GG3. (See FIGS. 4 and 5).

A small difference in the racial distribution was observed, however, this seems mainly driven by a larger number of men of non-Caucasian and non-African American descent, and when excluding this category, the differences were no longer significant. No significant differences were observed in the self-reported family history.

Biopsy-Positive Subsets. The main objective was to identify and to evaluate an immunocyte transcriptomic risk score that can segregate those men with indolent PCa that truly belong on AS, from those men with occult aggressive disease. To this end, the main validation endpoint was evaluated on biopsy-positive men only. A comparison between these men in biopsy-positive subsets, providing a surrogate for the AS setting, is presented in FIG. 5. The characteristics of both cohorts followed the same trends as was observed for the entire cohorts. Particularly for the training cohort, prostate volumes were slightly smaller than in the validation cohort, in line with the previously observed PSA levels. As a result, PSA density levels were similar between both cohorts. Somewhat counterintuitively, there is no clear sign of more aggressive disease in the less rigorously screened patients in the validation cohort. The proportion between the different GGs was not significantly different between both cohorts (p=0.6), in addition to the maximum percent cross-sectional involvement of a single core (p=0.7).

Model Performance in Biopsy-Positive Men. A regularized regression approach was used to avoid unnecessary model complexity and overfitting to the training set. The model with the best cross-validated AUC on the training cohort was selected, and subsequently independently validated on the validation cohort. This model for the AS setting was obtained by combining an immunocyte transcriptomic profile with PSA density and age, reaching an AUC of 0.73 (95% confidence interval (CI): 0.69-0.77).

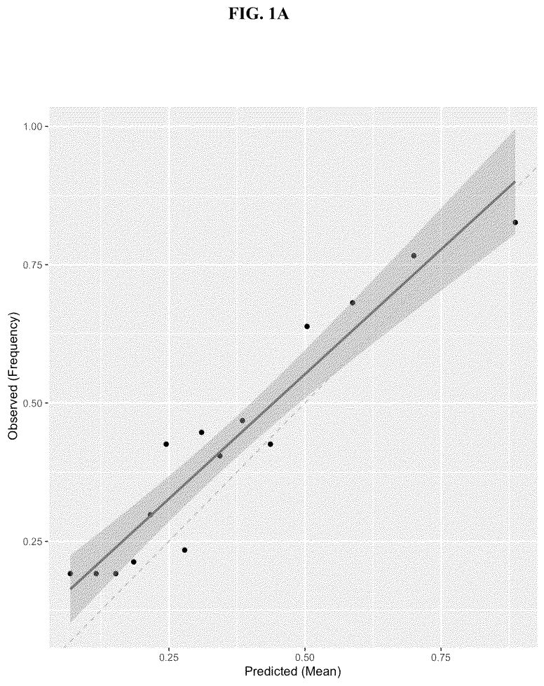

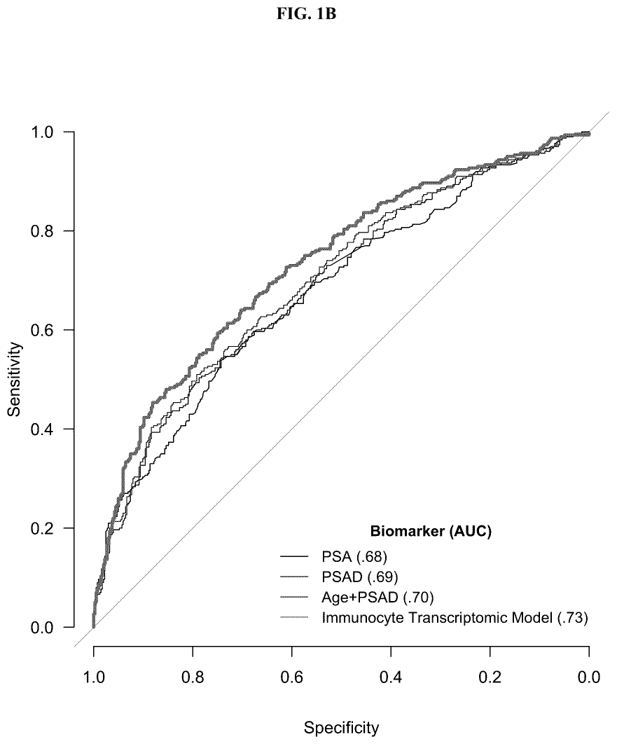

A calibration plot was generated (FIG. 1A) and showed that the observed risk aligned well with the predicted risk.

FIG. 1B further details that the model significantly outperformed (p<0.001) PSA density as a biomarker, which has an AUC of 0.69 (95% CI: 0.65-0.73; 95% CI of delta in AUC: 0.03-0.05), even when this was combined with age (AUC: 0.70, 95% CI: 0.66-0.74; p=0.006; 95% CI of delta in AUC: 0.03-0.04). PSA had only marginally lower performance compared to PSA density, with an AUC of 0.68 (95% CI: 0.64-0.72; p=0.003; 95% CI for delta in AUC: 0.04-0.07). The model was also compared with the prostate cancer prevention trial risk calculator with an AUC of 0.69 (95% CI: 0.65-0.73), which was significantly lower than that of the immunocyte transcriptomic model (p=0.035; 95% CI for delta in AUC: 0.02-0.06).

To give additional guidance in the patient's individualized risk, two cut points were defined, creating three categories of patients (See FIG. 3). First those at a 2.1-fold decreased risk than the average AS patient, associated with a negative predictive value (NPV) of 90% and representing 30% of an AS population. The middle category, constituting 40% of the cohort, represents those men at average risk of harboring aggressive disease. Finally, there are those with a highly elevated risk, at least 2.0-fold larger than the average AS patient, associated with a positive predictive value (PPV) of 50%.

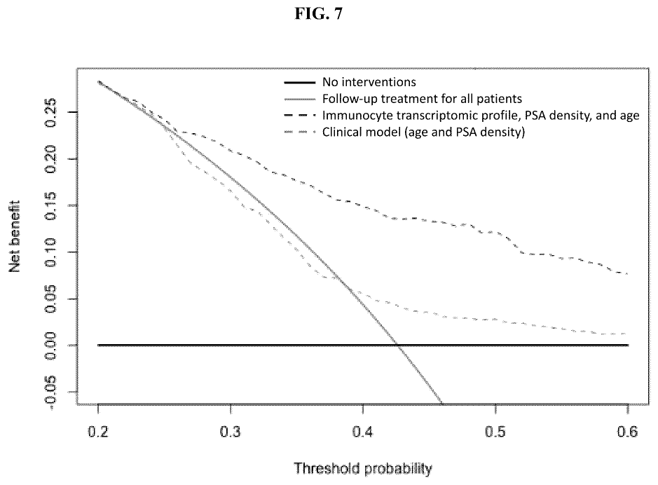

In order to further evaluate the clinical implications of the model, a decision curve analysis was performed (See FIG. 7). The decision curve analysis compared the overall model in the clinically significant risk range with the clinical model including only age and PSA density. While implementing this model could result in a positive impact on clinical practice, the clinical model (only age and PSA density) struggled to show a clear benefit over most of the range. The model combining the immunocyte transcriptomic profile with age and PSA density resulted in a definite clinical benefit, which was larger than the clinical model across the entire risk range.

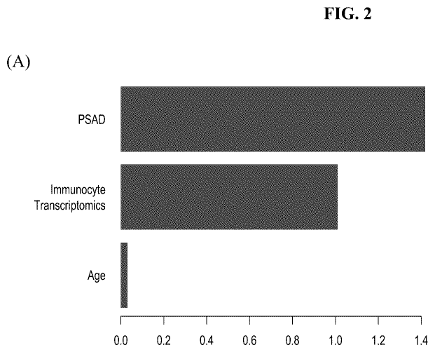

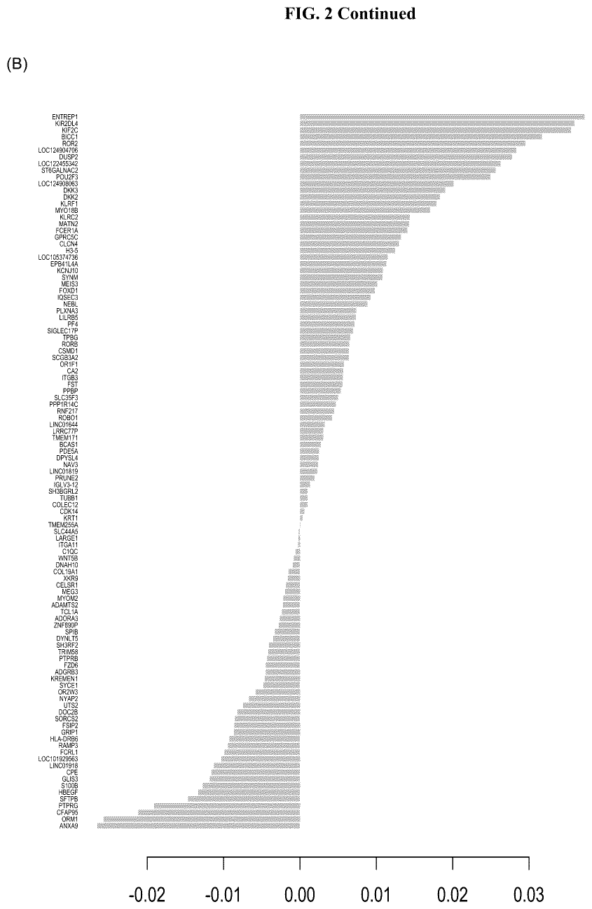

Biological Insights of the Immunocyte Transcriptomic Model. There was a strong contribution of the immunocyte transcriptomic part to the final model, accounting for 41% of the total performance (See FIG. 2). PSA density largely accounts for the remaining performance, with age just contributing 1.2%. A breakdown of the weights of 107 genes/biomarkers useful for a model of active surveillance (AS) of the disclosure is shown in FIG. 2, with respective weights shown for each gene/biomarker. Several KEGG pathways associated with cancer and immune response are linked to these genes, with the top ones being proteoglycans in cancer, basal cell carcinoma, phagosome, and WNT-signaling. Similar biological signals appear in WikiPathways, pointing towards the involvement of ncRNAs and lncRNAs in cancer-associated WNT-signaling. Thus, the disclosure provides an immune response link to the presence of aggressive PCa in an immunocyte transcriptomic signature.

### REFERENCES

1. Siegel R L, Miller K D, Fuchs H E, et al: Cancer statistics, 2022. C A Cancer J Clin 2022; 72: 7-33.
2. Loeb S, Bjurlin M A, Nicholson J, et al: Overdiagnosis and Overtreatment of Prostate Cancer. Eur Urol 2014; 65: 1046.
3. Moyer V A: Screening for prostate cancer: U.S. preventive services task force recommendation statement. Ann Intern Med 2012; 157.
4. Grossman D C, Curry S J, Owens D K, et al: Screening for prostate cancer US Preventive services task force recommendation statement. JAMA—Journal of the American Medical Association 2018; 319.
5. Kaul S, Wojno K J, Stone S, et al: Clinical outcomes in men with prostate cancer who selected active surveillance using a clinical cell cycle risk score. Per Med 2019; 16: 491-499.
6. Carroll P H and Mohler J L: NCCN Guidelines Updates: Prostate Cancer and Prostate Cancer Early Detection. Journal of the National Comprehensive Cancer Network 2018; 16: 620-623.
7. Anon: Active Surveillance—Michigan Urological Surgery Improvement Collaborative (MUSIC).
8. Vince R A, Sun Y, Mahal B, et al: The Impact of a Statewide Active Surveillance Initiative: A Roadmap for Increasing Active Surveillance Utilization Nationwide. Eur Urol 2022.
9. Shen F, Shinohara K, Kumar D, et al: Three-dimensional sonography with needle tracking: role in diagnosis and treatment of prostate cancer. J Ultrasound Med 2008; 27: 895-905.
10. Li W, Denton B T, Nieboer D, et al: Comparison of biopsy under-sampling and annual progression using hidden markov models to learn from prostate cancer active surveillance studies. Cancer Med 2020; 9: 9611-9619.
11. Mamawala M M, Rao K, Landis P, et al: Risk prediction tool for grade re-classification in men with favourable-risk prostate cancer on active surveillance. BJU Int 2017; 120: 25-31.
12. Jiang Y, Meyers T J, Emeka A A, et al: Genetic Factors Associated with Prostate Cancer Conversion from Active Surveillance to Treatment. HGG Adv 2022; 3.
13. Chen R C, Bryan Rumble R, Andrew Loblaw D, et al: Active Surveillance for the Management of Localized Prostate Cancer (Cancer Care Ontario Guideline): American Society of Clinical Oncology Clinical Practice Guideline Endorsement. J Clin Oncol 2016; 34: 2182-2190.
14. Hamdy F C, Donovan J L, Lane J A, et al: 10-Year Outcomes after Monitoring, Surgery, or Radiotherapy for Localized Prostate Cancer. N Engl J Med 2016; 375: 1415-1424.
15. Jiang Y, Meyers T J, Emeka A A, et al: Genetic Factors Associated with Prostate Cancer Conversion from Active Surveillance to Treatment. HGG Adv 2022; 3.
16. Allsbrook W C, Mangold K A, Johnson M H, et al: Interobserver reproducibility of Gleason grading of prostatic carcinoma: General pathologist. Hum Pathol 2001; 32: 81-88.
17. Wang M, Qi J, George A K, et al: The added influence of genomics and post-MRI confirmatory biopsy results to MRI results alone on medical decision making for men with favorable risk prostate cancer being considered for active surveillance. Prostate 2022; 82: 1068-1074.
18. de Bono J S, Guo C, Gurel B, et al: Prostate carcinogenesis: inflammatory storms. Nat Rev Cancer 2020; 20: 455-469.
19. van Neste L, Wojno K J, Henao R, et al: Evaluation of an RNAseq-Based Immunogenomic Liquid Biopsy Approach in Early-Stage Prostate Cancer. Cells 2021; 10.
20. Sturm M, Schroeder C and Bauer P: SeqPurge: Highly-sensitive adapter trimming for paired-end NGS data. BMC Bioinformatics 2016; 17: 1-7.
21. Dobin A, Davis C A, Schlesinger F, et al: STAR: ultrafast universal RNA-seq aligner. Bioinformatics 2013; 29: 15-21.
22. Liao Y, Smyth G K and Shi W: featureCounts: an efficient general purpose program for assigning sequence reads to genomic features. Bioinformatics 2014; 30: 923-930.
23. Anon: Babraham Bioinformatics—FastQC A Quality Control tool for High Throughput Sequence Data.
24. Ewels P, Magnusson M, Lundin S, et al: MultiQC: summarize analysis results for multiple tools and samples in a single report. Bioinformatics 2016; 32: 3047-3048.
25. Robinson M D and Oshlack A: A scaling normalization method for differential expression analysis of RNA-seq data. Genome Biol 2010; 11.
26. Fawcett T: An introduction to ROC analysis. Pattern Recognit Lett 2006; 27: 861-874.
27. DeLong E R, DeLong D M and Clarke-Pearson D L: Comparing the Areas under Two or More Correlated Receiver Operating Characteristic Curves: A Nonparametric Approach. Biometrics 1988; 44: 837.
28. Kanehisa M, Sato Y, Kawashima M, et al: KEGG as a reference resource for gene and protein annotation. Nucleic Acids Res 2016; 44: D457-D462.
29. Martens M, Ammar A, Riutta A, et al: WikiPathways: connecting communities. Nucleic Acids Res 2021; 49: D613-D621.
30. Hastie T, Tibshirani R and Friedman J: The Elements of Statistical Learning. 2009.
31. Willemse P P M, Davis N F, Grivas N, et al: Systematic review of active surveillance for clinically localized prostate cancer to develop recommendations regarding inclusion of intermediate-risk disease, biopsy characteristics at inclusion and monitoring, and surveillance repeat biopsy strategy. Eur Urol 2022; 81: 337-346.
32. Wang C, Zhang Y and Gao W Q: The evolving role of immune cells in prostate cancer. Cancer Lett 2022; 525: 9-21.
33. Ren X, Chen X, Zhang X, et al: Immune Microenvironment and Response in Prostate Cancer Using Large Population Cohorts. Front Immunol 2021; 12.
34. Chang R B and Beatty G L: The interplay between innate and adaptive immunity in cancer shapes the productivity of cancer immunosurveillance. J Leukoc Biol 2020; 108: 363-376.
35. Feng M, Jiang W, Kim B Y S, et al: Phagocytosis checkpoints as new targets for cancer immunotherapy. Nat Rev Cancer 2019; 19: 568-586.
36. Carlsson S, Benfante N, Alvim R, et al: Long-Term Outcomes of Active Surveillance for Prostate Cancer: The Memorial Sloan Kettering Cancer Center Experience. J Urol 2020; 203: 1122-1127.
37. Venderbos L D, Luiting H, Hogenhout R, et al: Interaction of MRI and active surveillance in prostate cancer: Time to re-evaluate the active surveillance inclusion criteria. Urol Oncol 2021.
38. Giganti F, Stavrinides V, Stabile A, et al: Prostate cancer measurements on serial MRI during active surveillance: it's time to be PRECISE. Br J Radiol 2020; 93.
39. Giganti F, Stabile A, Stavrinides V, et al: Natural history of prostate cancer on active surveillance: stratification by MRI using the PRECISE recommendations in a UK cohort. Eur Radiol 2021; 31: 1644-1655.
40. Giganti F, Aupin L, Thoumin C, et al: Promoting the use of the PRECISE score for prostate MRI during active surveillance: results from the ESOR Nicholas Gourtsoyiannis teaching fellowship. Insights Imaging 2022; 13.
41. Dominique G, Brisbane W G and Reiter R E: The utility of prostate MRI within active surveillance: description of the evidence. World J Urol 2022; 40: 71-77

It will be apparent to those skilled in the art that various modifications and variations can be made in the systems and methods of the present disclosure without departing from the spirit or scope thereof. Thus, it is intended that the present disclosure cover the modifications and variations of this disclosure provided they come within the scope of the appended claims and their equivalents.

What is claimed:

1. A method of identifying a patient with prostate cancer as a candidate for active surveillance of the prostate cancer comprising:
a) obtaining a blood sample from the patient with prostate cancer;
b) isolating CD14+ cells and CD2+ cells from the blood sample;
c) determining the gene expression level of 10 or more biomarkers selected from the group consisting of ENTREP1, KIR2DL4, KIF2C, BICC1, ROR2, LOC124904706, DUSP2, LOC122455342, ST6GALNAC2, POU2F3, LOC124908063, DKK3, DKK2, KLRF1, MYO18B, KLRC2, MATN2, FCER1A, GPRC5C, CLCN4, H3-5, LOC105374736, EPB41L4A, KCNJ10, SYNM, MEIS3, FOXD1, IQSEC3, NEBL, PLXNA3, LILRB5, PF4, SIGLEC17P, TPBG, RORB, CSMD1, SCGB3A2, OR1F1, CA2, ITGB3, FST, PPBP, SLC35F3, PPP1R14C, RNF217, ROBO1, LINC01644, LRRC77P, TMEM171, BCAS1, PDE5A, DPYSL4, NAV3, LINC01819, PRUNE2, IGLV3-12, SH3BGRL2, TUBB1, COLEC12, CDK14, KRT1, TMEM255A, SLC44A5, LARGE1, ITGA11, C1QC, WNT5B, DNAH10, COL19A1, XKR9, CELSR1, MEG3, MYOM2, ADAMTS2, TCL1A, ADORA3, ZNF890P, SPIB, DYNLT5, SH3RF2, TRIM58, PTPRB, FZD6, ADGRB3, KREMEN1, SYCE1, OR2W3, NYAP2, UTS2, DOC2B, SORCS2, FSIP2, GRIP1, HLA-DRB6, RAMP3, FCRL1, LOC101929563, LINC01918, CPE, GLIS3, S100B, HBEGF, SFTPB, PTPRG, CFAP95, ORM1, and ANXA9 in the CD14+ cells;
d) determining the expression level of the 10 or more selected biomarkers in the CD2+ cells;
e) normalizing the expression level of each of the selected biomarkers by determining the log ratio of CD14+ over CD2+ expression levels;
f) using the normalized expression level of each of the 10 or more selected biomarkers of step (e) to calculate an active surveillance risk score (ASRS) representing the probability that the patient is harboring aggressive prostate cancer; and
g) categorizing the patient into a group selected from very low risk, low/average risk, and high risk based on the patient's ASRS;
wherein when the ASRS is very low risk or low/average risk the patient with prostate cancer is identified as a candidate for active surveillance of the prostate cancer.

2. The method of claim 1, wherein the identified candidate for active surveillance of the prostate cancer enters active surveillance of the prostate cancer.

3. The method of claim 1, wherein the identified candidate for active surveillance of the prostate cancer remains on active surveillance of the prostate cancer.

4. The method of claim 1, further comprising obtaining one or more clinical data from the patient with prostate cancer selected from the group consisting of age, race, digital rectal exam (DRE), prostate volume, prostate density, family history, total prostate-specific antigen (PSA), PSA density (PSAD), tumor stage, tumor grade, tumor size, tumor visual characteristics, tumor growth, tumor thickness, tumor progression, tumor metastasis, tumor distribution within the body, odor, molecular pathology, genomics, and/or tumor angiograms.

5. The method of claim 4, wherein calculating an active surveillance risk score (ASRS) of step (f) further comprises utilizing the prostate-specific antigen (PSA) density of the patient with prostate cancer.

6. The method of claim 4, wherein calculating an active surveillance risk score (ASRS) of step (f) further comprises utilizing the age of the patient with prostate cancer.

7. The method of claim 4, wherein calculating an active surveillance risk score (ASRS) of step (f) further comprises utilizing the prostate-specific antigen (PSA) density and the age of the patient with prostate cancer.

8. The method of claim 1, wherein determining gene expression levels comprises using an amplification assay.

9. The method of claim 1, wherein determining gene expression levels comprises using polymerase chain reaction (PCR) analysis, sequencing analysis, electrophoretic analysis, restriction fragment length polymorphism (RFLP) analysis, Northern blot analysis, quantitative PCR, reverse-transcriptase-PCR analysis (RT-PCR), allele specific oligonucleotide hybridization analysis, comparative genomic hybridization, heteroduplex mobility assay (HMA), single strand conformational polymorphism (SSCP), denaturing gradient gel electrophisis (DGGE), RNAase mismatch analysis, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), surface plasmon resonance, Southern blot analysis, in situ hybridization, fluorescence in situ hybridization (FISH), chromogenic in situ hybridization (CISH), immunohistochemistry (IHC), microarray, comparative genomic hybridization, karyotyping, multiplex ligation-dependent probe amplification (MLPA), Quantitative Multiplex PCR of Short Fluorescent Fragments (QMPSF), microscopy, methylation specific PCR (MSP) assay, Hpaii tiny fragment Enrichment by Ligation-mediated PCR (HELP) assay, radioactive acetate labeling assays, colorimetric DNA acetylation assay, chromatin immunoprecipitation combined with microarray (ChiP-on-chip) assay, restriction landmark genomic scanning, Methylated DNA immunoprecipitation (MeDIP), molecular break light assay for DNA adenine methyltransferase activity, chromatographic separation, methylation-sensitive restriction enzyme analysis, bisulfite-driven conversion of non-methylated cytosine to uracil, methyl-binding PCR analysis, or a combination thereof.

10. The method of claim 1, wherein determining gene expression levels comprises using a sequencing assay.

11. The method of claim 10, wherein the sequencing assay is selected from the group consisting of direct sequencing, RNA sequencing (e.g., RNA-Seq (Illumina)), whole transcriptome shotgun sequencing, next generation sequencing, random shotgun sequencing, Sanger dideoxy termination sequencing, whole-genome sequencing, sequencing by hybridization, pyrosequencing, Ion Torrent, capillary electrophoresis, gel electrophoresis, duplex sequencing, cycle sequencing, single-base extension sequencing, solid-phase sequencing, high-throughput sequencing, massively parallel signature sequencing, sequencing using PacBio, Single Molecule Sequencing by Synthesis (SMSS) (Helicos), emulsion PCR, sequencing by reversible dye terminator, paired-end sequencing, near-term sequencing, exonuclease sequencing, primer walking, semiconductor sequencing, sequencing by ligation, short-read sequencing, single-molecule sequencing, sequencing-by-synthesis, real-time sequencing, reverse-terminator sequencing, nanopore sequencing, 454 sequencing, Clonal Single Molecule Array (Solexa), Genome Analyzer sequencing, Digital Gene Expression (Helicos), SOLID sequencing, Maxam-Gilbert sequencing, MS-PET sequencing, mass spectrometry, and a combination thereof.

12. The method of claim 1, further comprising (h) creating a report containing the active surveillance risk score (ASRS).

13. The method of claim 12, wherein the report containing the active surveillance risk score (ASRS) is utilized by the patient with prostate cancer and the patient's physician in a shared decision making process to determine the course of treatment or surveillance of the patient's prostate cancer.

14. The method of claim 13, wherein the report containing the active surveillance risk score (ASRS) is utilized by the patient with prostate cancer and the patient's physician in combination with one or more other guidelines or recommendations in a shared decision making process to determine the course of treatment or surveillance of the patient's prostate cancer.

15. The method of claim 1, comprising determining the gene expression level of 20 or more, 40 or more, 60 or more, 80 or more, or 100 or more selected biomarkers.

16. A method of characterizing prostate cancer aggressiveness in a patient with prostate cancer comprising:
a) obtaining a blood sample from the patient with prostate cancer;
b) isolating CD14+ cells and CD2+ cells from the blood sample;
c) determining the gene expression level of 10 or more biomarkers selected from the group consisting of ENTREP1, KIR2DL4, KIF2C, BICC1, ROR2, LOC124904706, DUSP2, LOC122455342, ST6GALNAC2, POU2F3, LOC124908063, DKK3, DKK2, KLRF1, MYO18B, KLRC2, MATN2, FCER1A, GPRC5C, CLCN4, H3-5, LOC105374736, EPB41L4A, KCNJ10, SYNM, MEIS3, FOXD1, IQSEC3, NEBL, PLXNA3, LILRB5, PF4, SIGLEC17P, TPBG, RORB, CSMD1, SCGB3A2, OR1F1, CA2, ITGB3, FST, PPBP, SLC35F3, PPP1R14C, RNF217, ROBO1, LINC01644, LRRC77P, TMEM171, BCAS1, PDE5A, DPYSL4, NAV3, LINC01819, PRUNE2, IGLV3-12, SH3BGRL2, TUBB1, COLEC12, CDK14, KRT1, TMEM255A, SLC44A5, LARGE1, ITGA11, C1QC, WNT5B, DNAH10, COL19A1, XKR9, CELSR1, MEG3, MYOM2, ADAMTS2, TCL1A, ADORA3, ZNF890P, SPIB, DYNLT5, SH3RF2, TRIM58, PTPRB, FZD6, ADGRB3, KREMEN1, SYCE1, OR2W3, NYAP2, UTS2, DOC2B, SORCS2, FSIP2, GRIP1, HLA-DRB6, RAMP3, FCRL1, LOC101929563, LINC01918, CPE, GLIS3, S100B, HBEGF, SFTPB, PTPRG, CFAP95, ORM1, and ANXA9 in the CD14+ cells;
d) determining the expression level of the 10 or more selected biomarkers in the CD2+ cells;
e) normalizing the expression level of each of the selected biomarkers by determining the log ratio of CD14+ over CD2+ expression levels;
f) using the normalized expression level of each of the 10 or more selected biomarkers of step (e) to calculate an active surveillance risk score (ASRS) representing the probability that the patient is harboring aggressive prostate cancer; and
g) categorizing the patient into a group selected from very low risk, low/average risk, and high risk based on the patient's ASRS.

17. The method of claim 16, wherein a patient with an ASRS of high risk is identified as being a patient with prostate cancer that is not a candidate for active surveillance.

18. The method of claim 16, wherein calculating an active surveillance risk score (ASRS) of step (f) further comprises utilizing the prostate-specific antigen (PSA) density and/or the age of the patient with prostate cancer.

19. A method of analyzing a blood sample from a patient with prostate cancer comprising:
a) obtaining a blood sample from the patient with prostate cancer;
b) isolating CD14+ cells and CD2+ cells from the blood sample;
c) determining the gene expression level of 10 or more biomarkers selected from the group consisting of ENTREP1, KIR2DL4, KIF2C, BICC1, ROR2, LOC124904706, DUSP2, LOC122455342, ST6GALNAC2, POU2F3, LOC124908063, DKK3, DKK2, KLRF1, MYO18B, KLRC2, MATN2, FCER1A, GPRC5C, CLCN4, H3-5, LOC105374736, EPB41L4A, KCNJ10, SYNM, MEIS3, FOXD1, IQSEC3, NEBL, PLXNA3, LILRB5, PF4, SIGLEC17P, TPBG, RORB, CSMD1, SCGB3A2, OR1F1, CA2, ITGB3, FST, PPBP, SLC35F3, PPP1R14C, RNF217, ROBO1, LINC01644, LRRC77P, TMEM171, BCAS1, PDE5A, DPYSL4, NAV3, LINC01819, PRUNE2, IGLV3-12, SH3BGRL2, TUBB1, COLEC12, CDK14, KRT1, TMEM255A, SLC44A5, LARGE1, ITGA11, C1QC, WNT5B, DNAH10, COL19A1, XKR9, CELSR1, MEG3, MYOM2, ADAMTS2, TCL1A, ADORA3, ZNF890P, SPIB, DYNLT5, SH3RF2, TRIM58, PTPRB, FZD6, ADGRB3, KREMEN1, SYCE1, OR2W3, NYAP2, UTS2, DOC2B, SORCS2, FSIP2, GRIP1, HLA-DRB6, RAMP3, FCRL1, LOC101929563, LINC01918, CPE, GLIS3, S100B, HBEGF, SFTPB, PTPRG, CFAP95, ORM1, and ANXA9 in the CD14+ cells;
d) determining the expression level of the 10 or more selected biomarkers in the CD2+ cells;
e) normalizing the expression level of each of the selected biomarkers by determining the log ratio of CD14+ over CD2+ expression levels; and
f) using the normalized expression level of each of the 10 or more selected biomarkers of step (e) to calculate an active surveillance risk score (ASRS) representing the probability that the patient is harboring aggressive prostate cancer.

20. The method of claim 19, further comprising (g) categorizing the patient with prostate cancer into a group selected from very low risk, low/average risk, and high risk based on the patient's ASRS.
